(19) United States
(12) Patent Application Publication
(10) Pub. No.: US 2020/0048468 A1
(43) Pub. Date: Feb. 13, 2020

(54) POLYMERIC HAIR COLOR COMPOSITION

(71) Applicant: Michael Milbocker, Holliston, MA (US)

(72) Inventor: Michael Milbocker, Holliston, MA (US)

(21) Appl. No.: 16/534,064

(22) Filed: Aug. 7, 2019

Related U.S. Application Data
(60) Provisional application No. 62/715,782, filed on Aug. 7, 2018.
(60) Provisional application No. 62/757,143, filed on Nov. 7, 2018.

(51) Int. Cl. C09B 69/10; A61K 8/87; A61Q 5/10

(52) U.S. Cl. CPC C09B 69/10; A61Q 5/10; A61K 8/87

(57) ABSTRACT

A polymeric hair colorant composition that may cure at ambient temperature, and is self-curable, may be obtained by attaching a protein bonding group to a polymeric backbone comprising chromophores. The protein bonding group may be an isocyanate or epoxy group pendant on the polymer backbone. The application of this self-curable polymeric hair colorant composition has neither organic solvent emission nor energy needed for curing.

### CROSS-REFERENCES TO RELATED APPLICATIONS

This application claims benefit of the following patent application(s) which is/are hereby incorporated by reference: U.S. Provisional Patent App. No. 62/715,782 filed on Aug. 7, 2018; and U.S. Provisional Patent App. No. 62/757,143 filed on Nov. 7, 2018.

A portion of the disclosure of this patent document contains material that is subject to copyright protection. The copyright owner has no objection to the reproduction of the patent document or the patent disclosure, as it appears in the U.S. Patent and Trademark Office patent file or records, but otherwise reserves all copyright rights whatsoever.

### STATEMENT REGARDING FEDERALLY SPONSORED RESEARCH OR DEVELOPMENT

Not Applicable

### REFERENCE TO SEQUENCE LISTING OR COMPUTER PROGRAM LISTING APPENDIX

Not Applicable

### BACKGROUND OF THE INVENTION

The present invention relates generally to a polymeric hair colorant composition.

More particularly, this invention pertains to a polymeric hair colorant composition that cures at ambient temperature, is self-curable, and is obtained by attaching a protein bonding group to a polymeric backbone comprising chromophores.

Consumers desire to use cosmetic and personal care compositions that enhance the appearance of keratin fibers, such as hair, by changing the color of the hair and/or by imparting various properties to hair, for example, shine, tensile strength, increased hair shaft diameter, and hair volume. The process of changing the color of hair can involve depositing an artificial color onto the hair, which provides a different shade or color to the hair, and/or lifts the color of the hair.

Currently, conventional hair coloring products may include permanent dye compositions comprising oxidation dye precursors, which are also known as primary intermediates or couplers. These oxidation dye precursors are colorless or weakly colored compounds which, when combined with oxidizing agents, give rise to colored complexes by a process of oxidative condensation.

In general, hair colorants require the presence of an alkalizing agent such as ammonia or an ammonia gas generating compound and/or an amine or ammonium-based compound in amounts sufficient to make such compositions alkaline. The alkalizing agent causes the hair shaft to swell, thus allowing the small oxidative dye molecules to penetrate the cuticle and cortex before the oxidation condensation process is completed. The resulting larger-sized colored complexes from the oxidative reaction are then trapped inside the hair fiber, thereby permanently altering the color of the hair. While such hair dyeing and/or color lifting compositions can effectively alter the color of hair, these compositions can damage the hair fibers and/or irritate the scalp due to having excessively high levels of alkalinity.

In addition, conventional permanent hair coloration compositions are two-part, resulting in messy preparation and application. The two parts require considerable time to work in combination. Consequently, there is a need for a one-part, fast acting hair colorant.

In an effort to reduce or avoid the drawbacks above, as well as to improve the cosmetic performance of the hair coloration compositions, the use of new and additional ingredients and novel combinations of ingredients are continuously sought. However, the choice of ingredients or combinations of ingredients could pose difficulties insofar as they cannot be detrimental to other cosmetic attributes such as ease and uniformity of application, rheology or viscosity properties, stability of the compositions, color deposit, and target shade formation. By decreasing the quality of these attributes, the result may provide more disadvantages such as increased damage or less healthy hair. It is therefore desirable to provide the consumer with compositions and methods that can change the color of hair and additionally, deposit color onto hair in an efficient or improved manner, provide cosmetic advantages such as shine, conditioning, and healthy appearance, while preventing excess damage to the hair.

### BRIEF SUMMARY OF THE INVENTION

The present invention provides various one-part hair coloration treatments that may comprise a cross linking reaction of a functionalized polymer with hair. Inone embodiment, a self-curable system of polymeric color may be disclosed comprising a reactive amino group to obtain chemical linkage between the reactive amino group of the color polymer and protein of hair.

In one embodiment, a single pack, ambient temperature, self-curable, nonaqueous borne-based polymeric hair colorant may be obtained by this invention by use of a dye attached to an amphiphilic backbone with at least one pendant amino group and dispersed in a nonaqueous carrier. The polymeric hair color solution may be stable during storage at a pH in the range of 4.0 to 10.0 and may self-cure after application to dry or wet hair. Curing comprises bonding to the protein in hair, as well as self-crosslinking, both attaching to and sealing the hair cuticle. These self-cured polymeric hair colors after curing may be water and solvent resistant and may dry at ambient temperature.

### BRIEF DESCRIPTION OF THE SEVERAL VIEWS OF THE DRAWINGS

FIG. 1 is a general structure for the polymeric backbone of the hair colorant composition of the present invention.

FIG. 2 is a general structure of a reactive group of the present invention.

FIG. 3 is a general formula of the polymeric hair dye.

FIG. 4 is a general structure for one embodiment of the dye moiety of the present invention.

FIG. 4a is a structure that may be substituted for “A” of FIG. 4.

FIG. 4b is a structure that may be substituted for “A” of FIG. 4.

FIG. 4c is a structure that may be substituted for “A” of FIG. 4.

FIG. 5 is a structure for one embodiment of the dye moiety of the present invention.

FIG. 6 is a structure for one embodiment of the dye moiety of the present invention.

FIG. 7 is a structure for one embodiment of the dye moiety of the present invention.

FIG. 8 is a structure for one embodiment of the dye moiety of the present invention.

FIG. 9 is a reaction for the synthesis linking a dye moiety to a functional group.

FIG. 10 is a reaction for the synthesis linking a dye moiety to a functional group.

FIG. 11 is a general formula for a diisocyanate of the present invention.

FIG. 11a is the structure for toluene-2,4-diisocyanate.

FIG. 11b is the structure for isophorone diisocyanate.

FIG. 12 is a general synthesis reaction for combining a dye moiety with a diisocyanate.

FIG. 13 is a general reaction for a polymer hair colorant reacted into a polyurea.

FIG. 14 is a general reaction for a polymer hair colorant reacted into a polyurethane.

FIG. 15 is a general formula for diamines.

FIG. 15a is the structure for toluene-2,4-diamine.

FIG. 15b is the structure for isophorone diamine.

FIG. 16 is a general formula for diols.

FIG. 16a is the structure for toluene-2,4-diol.

FIG. 16b is the structure for isophorone diol.

FIG. 17 is a general formula and structure for a polyol of the present invention.

### DETAILED DESCRIPTION OF THE INVENTION

Hair coloring products (water or solvent soluble) or for that matter, any dye, having pendant hydroxyl or amine groups which may be attached to an amphiphilic backbone comprising at least one pendant amino group may form a self-curing polymeric hair coloration.

The backbone polymers provided in this invention are preferably amphiphilic, though other polymers may also be used. The backbone polymer may be selected from one or more than one of the group of carboxyl groups containing polyurethane (PU) (hereinafter referred to as “PU based polymer:), epoxy resin (hereinafter referred to as “epoxy based polymer”), poloxamer, polyethylene glycol, polylactic acid, and modified acrylate copolymer resin having an epoxy group (hereinafter referred to as “acrylate based copolymer”). These polymers may offer a polymer moiety for supporting various dyes to be used in this invention. In some embodiments, the polymer backbone may contain internal ionic carboxyl groups that may stabilize nonaqueous carrier-borne polymer dispersions.

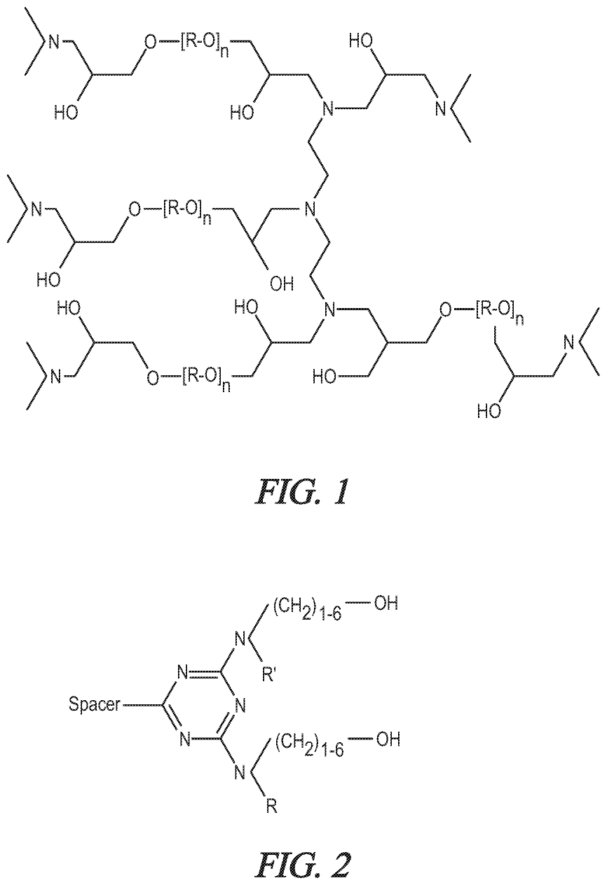

In one embodiment, the polymer backbone may have the general structure as illustrated in FIG. 1.

Aliphatic epoxy resins may be formed by glycidylation of aliphatic alcohols or polyols. The resulting resins may be monofunctional (e.g. dodecanol glycidyl ether), difunctional (butanediol diglycidyl ether), or have higher functionality (e.g. trimethylolpropane triglycidyl ether). These resins may display low viscosity at room temperature (10-200 mPa's) and may be referred to as reactive diluents. They may rarely be used alone, but often may be employed to modify (reduce) the viscosity of other epoxy resins. This has led to the term ‘modified epoxy resin’ to denote those containing viscosity-lowering reactive diluents. A related class is cycloaliphatic epoxy resins, which contain one or more cycloaliphatic rings in the molecule (e.g. 3,4-epoxycyclohexylmethyl-3,4-epoxycyclohexane carboxylate). This class may also display low viscosity at room temperature but can offer significantly higher temperature resistance than the aliphatic epoxy diluents. However, reactivity may be rather low compared to other classes of epoxy resin, and high temperature curing using suitable accelerators may be required.

PU based polymers having an NCO-terminated PU pre-polymer may be obtained from the reaction of polyols with one or more diisocyanates. An amine or hydroxyl containing dye may be selected to react toward the NCO-terminated PU pre-polymer and results in a formation of a polymeric hair dye with a PU polymer moiety.

An epoxy-based polymer may be obtained from a commercially available epoxy resin with EEW (epoxy equivalent weight) 780-850 (for example, trade name of NPES-904 available from Nan Ya Plastics Corporation, Taiwan) as a starting material. Its secondary hydroxyl group may be reacted with one or more diisocyanates. An amino containing dye, such as acid dye or direct dye, may be selected to react with the epoxide group or isocyanate group to form an epoxy based polymeric hair dye.

In some embodiments, a polymeric composition comprising one or more colored urea or urethane pendant groups may be used. Colored hair containing colorants is known to lose color when exposed to solar radiation for extended times. Hair colorants may degrade by shifting their reflectance or transmission spectrum, often on exposure to solar radiation within days.

Prior art hair colorants have varied chromophore compositions which are selected depending upon the dye color and the use of the pigment. The colorants are not covalently bonded to a polymer backbone to fix associations of the various different chromophores and tend to agglomerate by type leading to inhomogeneous distribution of colorants within the carrier. Non-covalently bonded colorants also tend to fade, bleed, or shift in apparent color. The color shift occurs by the unequal fading of different color components.

There are at least three different ways to covalently attach a dye molecule to a polymer backbone. The first may comprise grafting the dye molecule onto a reactive side group of the polymer backbone. The second way may comprise capping the ends of a polymer backbone with a monofunctional dye molecule reactive to the polymer chain. And the third way may comprise reacting a difunctional dye monomer together with other monomers into a polymer chain. All three types may be used in the present invention.

For example, in one embodiment, the polymeric hair dye may be obtained by the chain extension of a PU prepolymer with a colorant. The colorant may be difunctional, either with two functional groups reactive to isocyanate, e.g. hydroxyl or amine, or two isocyanate groups. In the first step of a two-step process, a PU prepolymer may be synthesized, and in the second step the polymer chain may be extended with the functionalized dye molecules. In the polymerization process the final product may contain a sufficient number of terminal isocyanate groups to provide in situ bonding to hair.

In another embodiment, the polymeric hair colorant may be formed by dye monomers co-reacted into a polyurethane. The dye monomers may have at least one group reactive to isocyanate attached to a dye moiety by a link. The link may be either a direct bond, oxygen, an amine, or an amide group. The reactive group may further be defined as being a very specific group having at least two hydroxyl groups. Referring to FIG. 2, one embodiment of this specific group is of the general formula where R and R′ may each independently be hydrogen, C.sub.1-4 alkyl, or further C. sub.1-6-omega-hydroxyalkyl groups.

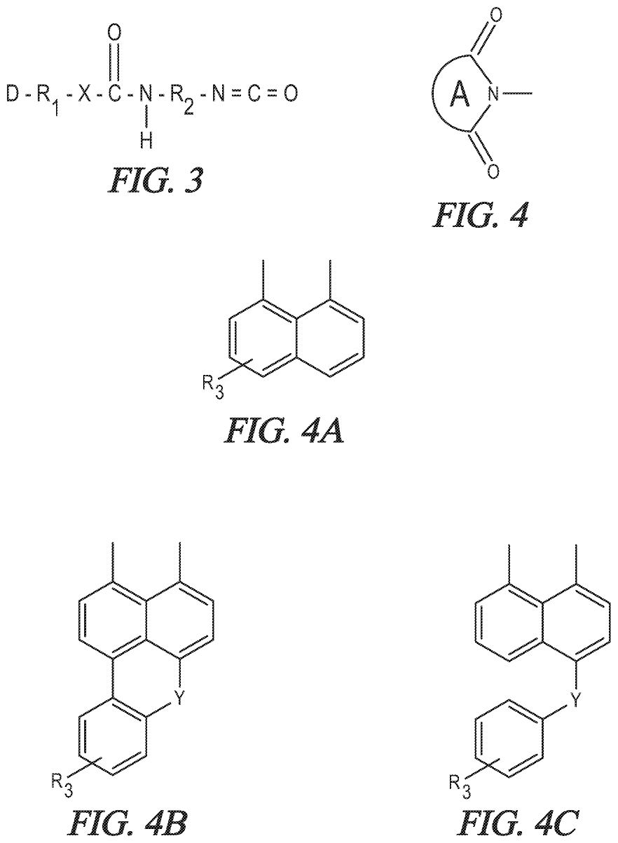

One embodiment may be achieved by a composition comprising one or more dye groups reacted to, or to be incorporated into, the backbone of a multi-armed polymer. In this embodiment the dye moiety is attached to the polymer backbone by a link containing a urea or urethane group. The polymeric hair dye may be of the general formula given in FIG. 3. The general formula may comprise R.sub.1 which may be chosen from C.sub.1-12 alkylene, C.sub.6-10 arylene, (C.sub.6-10) aryl-(C.sub.1-6) alkylene or (C.sub.1-6) alkyl-(C.sub.6-10) arylene, —(C.sub.1-6 alkylene-O-).sub.n-C.sub.1-6 alkylene- with “n” being an integer from 0 to 6; the alkylene and/or arylene radicals optionally being substituted by hydroxyl, C.sub.1-6 alkyl, C.sub.1-6 alkoxyl, C.sub.6-10 aryloxy or halogen; “X” may be chosen from oxygen or NR′ with R′ being hydrogen, C.sub.1-6 alkyl, C.sub.6-10 aryl, (C.sub.6-10) aryl-(C.sub.1-6) alkyl or (C.sub.1-6) alkyl-(C.sub.6-10) aryl, the alkyl and/or aryl radicals optionally being substituted by hydroxyl, C.sub.1-6 alkoxyl, C.sub.6-10 aryloxy or halogen; R.sub.2 may be chosen from C.sub.1-12 alkylene, C.sub.5-6 cycloalkylene, C.sub.6-10 arylene, (C.sub.6-10) aryl-(C.sub.1-6) alkylene or (C.sub.1-6) alkyl-(C.sub.6-10) arylene, —(C.sub.1-6 alkylene-O-).sub.n-C.sub.1-6 alkylene- with “n” being an integer from 0 to 6; the alkylene, cycloalkylene and/or arylene radicals optionally being substituted by hydroxyl, C.sub.1-6 alkyl, C.sub.1-6 alkoxyl, C.sub.6-10 aryloxy or halogen, and D may be a dye moiety of the general formulae given in FIGS. 4 to 8.

Referring to FIGS. 4-8, A may be comprised of any one of the depicted structures, wherein A may be chosen from a substituted or unsubstituted fused aromatic or heterocyclic ring system, preferably of the general formula depicted in FIG. 4a, 4b or 4c, wherein R.sub.3 being hydrogen, halogen, NR.sub.4R.sub.5, R.sub.5O or R.sub.5S; R.sub.4 may be chosen from hydrogen, C.sub.1-6 alkyl, C.sub.6-10 aryl, (C.sub.6-10) aryl-(C.sub.1-6) alkyl or (C.sub.1-6) alkyl-(C.sub.6-10) aryl, the alkyl and/or aryl radicals optionally being substituted by hydroxyl, C.sub.1-6 alkoxyl, C.sub.6-10 aryloxy or halogen; R.sub.5 being C.sub.1-6 alkyl, C.sub.6-10 aryl, (C.sub.6-10) aryl-(C.sub.1-6) alkyl or (C.sub.1-6) alkyl-(C.sub.6-10) aryl, the alkyl and/or aryl radicals optionally being substituted by hydroxyl, C.sub.1-6 alkoxyl, C.sub.6-10 aryloxy or halogen; and Y may be chosen from sulphur, oxygen, or NR.sub.4, with R.sub.4 having the meaning given above wherein the Ring B may be annelated in 3,4-position with a group of the formula —NR.sub.6(CO).sub.m-NR.sub.7- or —O—CO—NR.sub.6-, wherein “m” may be 1 or 2, R.sub.6 and R.sub.7 may be independently hydrogen, C.sub.1-6 alkyl, C.sub.6-10 aryl, (C.sub.6-10) aryl-(C.sub.1-6)alkyl or (C.sub.1-6)alkyl-(C.sub.6-10)aryl, the alkyl and/or aryl radicals optionally being substituted by amino, C.sub.1-6 alkylamino, C.sub.6-10 cycloalkylamino, hydroxyl, C.sub.1-6 alkoxyl, C.sub.6-10 aryloxy or halogen.

Especially preferred are those of the general formula given in FIG. 4 where A may be of the general formula of FIG. 4b, with Y being sulphur and R.sub.3 being hydrogen and those of the general formula given in FIG. 7 with R.sub.3 being hydrogen.

In one embodiment, the links R.sub.1 and R.sub.2 may independently be chosen from C.sub.2-6 alkylene, C.sub.6 cycloalkylene, —(C.sub.1-6 alkylene-O—).sub.n-C.sub.1-6-alkylene- with “n” being 1, 2 or 3, and C.sub.6 arylene optionally substituted. In a further embodiment, the links R.sub.1 and R.sub.2 may independently be chosen from C.sub.2, C.sub.3 or C.sub.6 alkylene, -(ethylene-O-).sub.n-ethylene- with “n” being 2. X may comprise oxygen or NR′ with R′ comprising hydrogen or methyl.

In some embodiments, the content of the polymerizable polymer hair colorant monomer in a hair colorant preparation may be from 10 to 100, more preferably from 10 to 50, and most preferably between 20 and 30 percent by weight based on the total weight of the hair colorant preparation.

The intermediates for the synthesis of the polymeric hair colorant of the formula of FIG. 3 with D being of one of the formulae depicted in FIG. 4 may be obtained by the condensation of the dicarboxylic anhydride of the respective dye moiety with an aminoalcohol or a diamine comprising the respective link according to the general formula depicted in FIG. 9 in a polar aprotic solvent according to the reaction depicted in FIG. 1 wherein A, R.sub.1, and X may be as defined above.

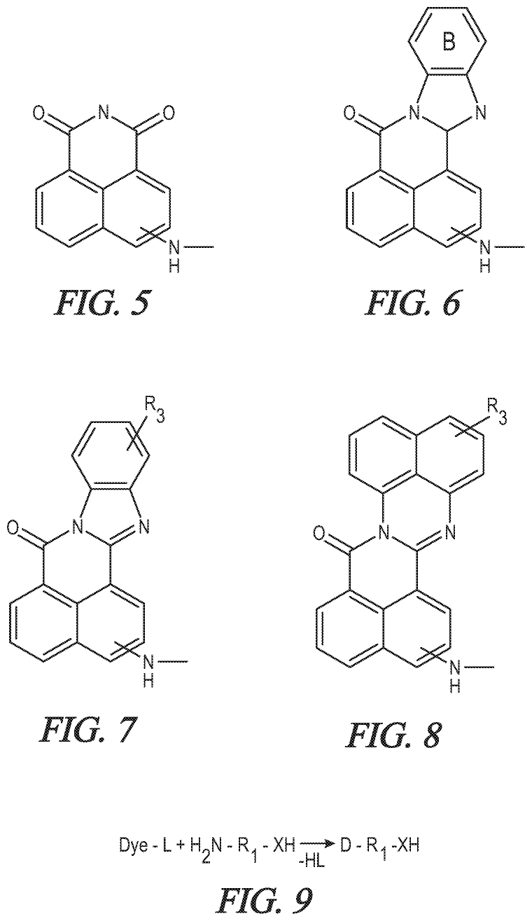

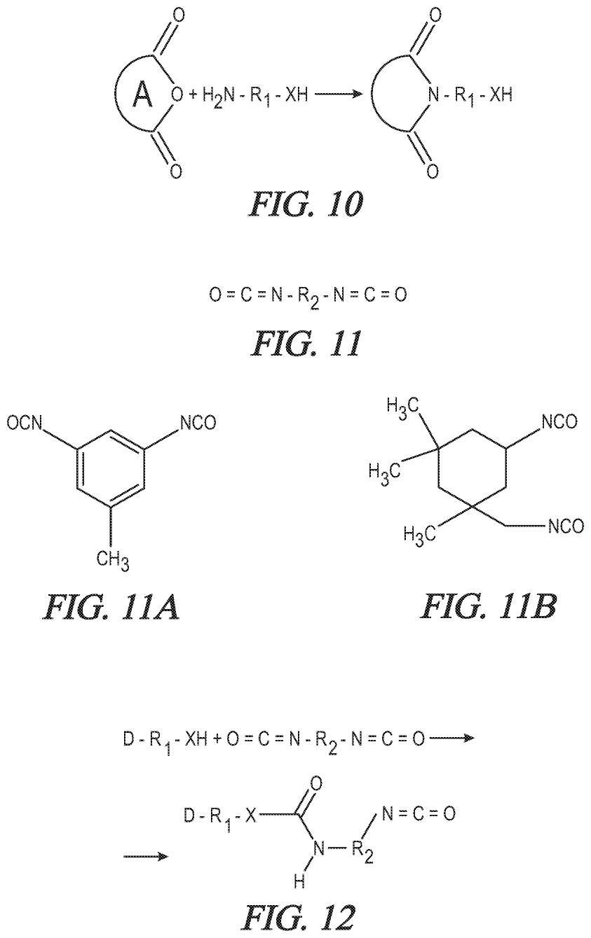

The intermediates for the synthesis of the polymeric hair colorant of the formula of FIG. 3 with D being one of the formulae depicted in FIGS. 5 to 8 may be obtained by the reaction of the polymeric hair colorant molecule functionalized with a leaving group (L), e.g. chlorine or bromine, with an amino alcohol or diamine comprising the respective link according to the general formula depicted in FIG. 9. The reaction is depicted in FIG. 10.

In some embodiments, the hydroxyl or amino group on the free end of the link may further be functionalized with diisocyanate moieties of the general formula depicted in FIG. 11. The final product may be obtained in high yield via the disclosed process.

The synthesis according to one embodiment is depicted in FIG. 12, wherein D, R.sub.1, R.sub.2 and X may be as defined above.

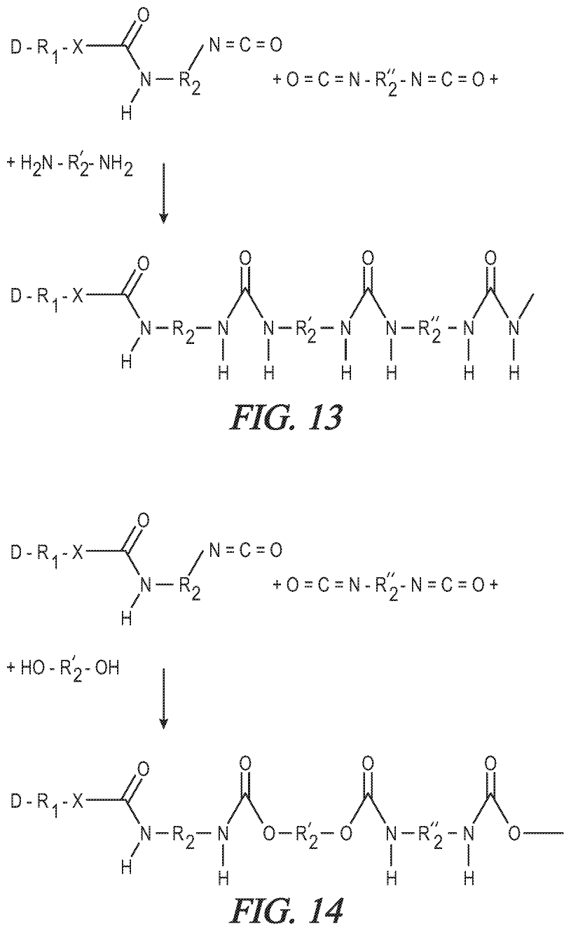

In another embodiment, the synthesis for the polymer hair colorant being reacted into a polyurea may be shown in FIG. 13, wherein D, R.sub.1, R.sub.2 and X may be defined as above; and R.sub.2′ and R.sub.2″ independently may be chosen from C.sub.1-12 alkylene, C.sub.5-6 cycloalkylene, C.sub.6-10 arylene, (C.sub.6-10) aryl-(C.sub.1-6) alkylene or (C.sub.1-6) alkyl-(C.sub.6-10) arylene, -(C.sub.1-6-alkylene-O-).sub.n-C.-sub.1-6 alkylene- with “n” being an integer from 0 to 6; the alkylene, cycloalkylene and/or arylene radicals optionally being substituted by hydroxyl, C.sub.1-6 alkyl, C.sub.1-6 alkoxyl, C.sub.6-10 aryloxy or halogen.

Another embodiment showing the synthesis for the polymeric hair colorant being reacted into a polyurethane may be shown in FIG. 14, wherein D, R.sub.1, R.sub.2, R.sub.2′ R.sub.2″ and X may be defined as above.

Diisocyanates which may be used may be chosen from 1,4-tetramethylene diisocyanate, 1,6-hexamethylene diisocyanate, 1-isocyanato-3-isocyanatomethyl-3,5,5-trimethyl-cyclohexane (also referred to as isophorone diisocyanate), 4,4′-diisocyanato-dicyclohexylmethane, phenylene-1,4-diisocyanate, 2,4- and 2,6-toluene diisocyanate, naphthylene-1,5-diisocyanate, 3,3′-bitoluene 4,4′-diisocyanate, diphenylmethane-4,4′-diisocyanate, 3,3′-dimethyl diphenylmethane 4.4′-diisocyanate, meta phenylene diisocyanate, 2,4-tolylene diisocyanate dimer and dianisidine diisocyanate, or salts thereof.

Some embodiments may include preferred diisocyanates (such as compositions in FIG. 11) which may be chosen from C.sub.6-12 alkylene diisocyanates, toluene-2,4-diisocyanate (FIG. 11a) or 1-isocyanato-3-isocyanatomethyl-3,5,5-trimethylcyclohexane (also referred to as isophorone diisocyanate) (FIG. 11b).

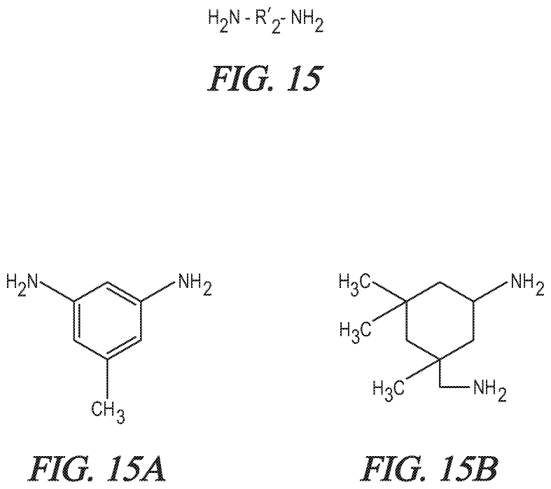

Some embodiments may include preferred diamines (such as compositions in FIG. 15) which may be chosen from C.sub.6-12 alkylene diamine, C.sub.5-6 cycloalkylene diamine, toluene-2,4-diamine (FIG. 15a) or 1-amino-3-aminomethyl-3,5,5-trimethylcyclohexane, also referred to as isophorone diamine (FIG. 15b).

Some embodiments may include preferred diamines (such as compositions in FIG. 15) which may be chosen from 1,2-ethylenediamine, 1,4-tetramethylene diamine, 1,6-hexamethylenediamine, trimethyl hexamethylene diamine, 1,4-diaminocyclohexane, toluene-2,4-diamine (FIG. 15a) or mino-3-aminomethyl-3,5,5-trimethylcyclohexane, also referred to as isophorone diamine (FIG. 15b).

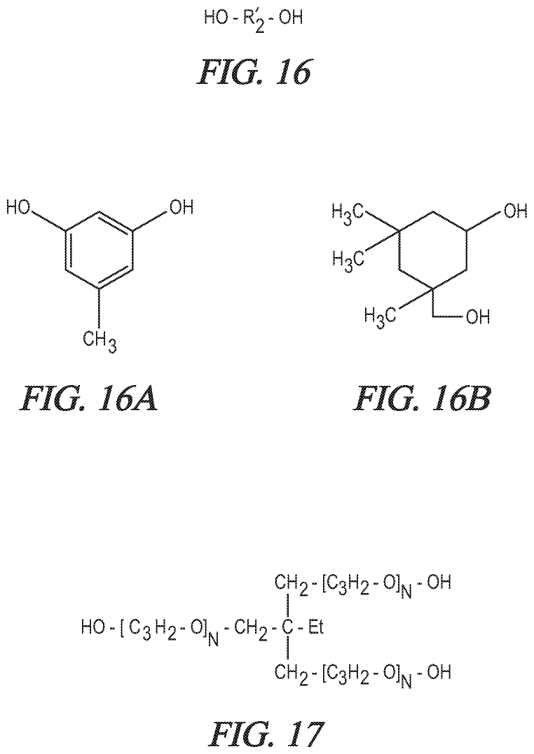

Some embodiments may include preferred diols (such as compositions in FIG. 16) which may be chosen from C.sub.6-12 alkyl enediols, toluene-2,4-diol (FIG. 16a) or 1-hydroxy-3-hydroxymethyl-3,5,5-trimethylcyclohexane (also referred to as isophorone diol) (FIG. 16b). The diols may be reacted by triols to form polyols. Polyols may be of the composition depicted in FIG. 17.

The reaction temperature may be in the range from 0 to 100.degree. C., preferably around 80.degree C.

A dispersing agent or solvent may be present in an amount from 0 to 20%, preferably around 5% by weight of the reaction mixture.

The following examples illustrate various embodiments of the invention. Unless otherwise specified, parts and percentages used in the examples are on a weight to weight basis.

In one embodiment, the polymeric hair colorant may be mixed with one or more anhydrous organic or inorganic solvents. Suitable organic solvents for use in the oxidizing composition may include ethanol, isopropyl alcohol, propanol, benzyl alcohol, phenyl ethyl alcohol, glycols and glycol ethers, such as propylene glycol, hexylene glycol, ethylene glycol monomethyl, monoethyl or monobutyl ether, propylene glycol and its ethers, such as propylene glycol monomethyl ether, butylene glycol, dipropylene glycol, diethylene glycol alkyl ethers, such as diethylene glycol monoethyl ether and monobutyl ether, ethylene glycol, propylene glycol, butylene glycol, hexylene glycol, propanediol, glycerin, hydrocarbons such as straight chain hydrocarbons, mineral oil, polybutene, propylene carbonate, hydrogenated polyisobutene, hydrogenated polydecene, polydecene, squalane, petrolatum, isoparaffins, any dried botanically derived oils, their salts, and mixtures thereof.

The organic solvents for use according to the present disclosure may be volatile or non-volatile compounds. The organic solvent may, for example, be present in an amount ranging from about 0.5% to about 70% by weight, such as from about 2% to about 60% by weight, such as from about 5 to about 50% by weight, relative to the total weight of the polymeric hair colorant composition.

In various embodiments, one or more chromophores may be present in the polymeric hair colorant composition. In a further embodiment one or more polymeric hair colorants may be mixed before use. And in yet a further embodiment one or more colorants may be present in the polymeric hair colorant and one or more other colorants may be present in a separate composition that may be mixed with the polymeric hair colorant before use.

In various embodiments, the chromophore can be chosen from oxidative dye precursors, to which hydroxyl groups or amine groups may optionally be attached, which may then be oxidized before attachment to the polymeric backbone, direct dyes, pigments, and mixtures thereof.

The oxidation dyes may be chosen from one or more oxidation bases optionally combined on one or more arms of the polymeric backbone. By way of example, the oxidation bases may be chosen from para-phenylenediamines, bis(phenyl)alkylenediamines, para-aminophenols, ortho-aminophenols, meta-aminophenols, and heterocyclic bases, and the salts thereof.

Among the para-phenylenediamines that may be selected, for example, are para-phenylenediamine, para-toluenediamine (toluene-2,5-diamine), chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(.beta.-hydroxyethyl)-para-phenylenediamine, 4-N,N-bis(.beta.-hydroxyethyl)amino-2-methylaniline, 4-N,N-bis(.beta.-hydroxyethyl)amino-2-chloroaniline, 2-.beta.-hydroxyethyl-para-phenylenediamine, 2-methoxymethyl-para-phenylenediamine, 2-fluoro-para-phenylenediamine, 2-isopropyl-para-phenylenediamine, N-(.beta.-hydroxypropyl)-para-phenylenediamine, 2-hydroxymethyl-para-phenylenediamine, N,N-dimethyl-3-methyl-para-phenylenediamine, N-ethyl-N-(.beta.-hydroxyethyl)-para-phenylenediamine, N-(.beta.,.gamma.-dihydroxypropyl)-para-phenylenediamine, N-(4′-aminophenyl)-para-phenylenediamine, N-phenyl-para-phenylenediamine, 2-.beta.-hydroxyethoxy-para-phenylenediamine, 2-.beta.-acetylamino ethyl oxy-para-phenylenediamine, N-(.beta.-methoxyethyl)-para-phenylenediamine, 4-aminophenylpyrrolidine, 2-thienyl-para-phenylenediamine, 2-.beta.-hydroxyethylamino-5-aminotoluene and 3-hydroxy-1-(4′-aminophenyl)pyrrolidine, hydroxypropyl bis(n-hydroxyethyl-p-phenylenediamine, and the salts thereof.

Para-phenylenediamines may be chosen from para-phenylenediamine, para-toluenediamine, 2-isopropyl-para-phenylenediamine, 2-.beta.-hydroxyethyl-para-phenylenediamine, 2-.beta.-hydroxyethoxy-para-phenylenediamine, 2,6-dimethyl-para-phenylenediamine, 2,6-diethyl-para-phenylenediamine, 2,3-dimethyl-para-phenylenediamine, N,N-bis(.beta.-hydroxyethyl)-para-phenylenediamine, 2-chloro-para-phenylenediamine and 2-.beta.-acetylamino ethyl oxy-para-phenylenediamine, and the salts thereof.

Bis(phenyl)alkylenediamines may be chosen from N,N′-bis(.beta.-hydroxyethyl)-N,N′-bis(4′-aminophenyl)-1,3-diaminopropano-1, N,N′-bis(.beta.-hydroxyethyl)-N,N′-bis(4′-aminophenyl)ethylenediamine, N,N′-bis(4-aminophenyl)tetramethylenediamine, N,N′-bis(.beta.-hydroxyethyl)-N,N′-bis(4-aminophenyl)tetramethylenediamine, N,N′-bis(4-dimethylaminophenyl)tetramethylene diamine, N,N′-bis(ethyl)-N,N′-bis(4′-amino-3′-methylphenyl)ethylenediamine and 1,8-bis(2,5-diamino phenoxy)-3,6-dioxaoctane, and the salts thereof.

Para-aminophenols may be chosen from para-aminophenol, 4-amino-3-methylphenol, 4-amino-3-fluorophenol, 4-amino-3-chlorophenol,4-amino-3-hydroxymethyl phenol, 4-amino-2-methylphenol, 4-amino-2-hydroxymethyl phenol, 4-amino-2-methoxymethyl phenol, 4-amino-2-aminomethyl phenol,4-amino-2-(.beta.-hydroxyethyl aminomethyl)phenol and 4-amino-2-fluorophenol, and the salts thereof.

Ortho-aminophenols may be chosen from 2-aminophenol, amino-5-methylphenol, 2-amino-6-methylphenol and 5-acetamido-2-aminophenol, and the salts thereof.

Among the meta-aminophenols, 3-aminophenol and salts thereof, may be selected for use.

Heterocyclic base may be selected from pyridine derivatives, pyrimidine derivatives and pyrazole derivatives.

Pyridine derivatives may be selected from 2,5-diaminopyridine, 2-(4-methoxyphenyl)amino-3-aminopyridine and 3,4-diaminopyridine, and the salts thereof.

Other pyridine oxidation bases that may be used in the present disclosure are the 3-amino pyrazolo[1,5-a]pyridine oxidation bases or the salts thereof. Examples may include pyrazolo[1,5-a]pyridin-3-ylamine, 2-acetylamino pyrazolo[1,5-a]pyridin-3-ylamine, 2-morpholin-4-yl pyrazolo[1,5-a]pyridin-3-ylamine, aminopyrazolo[1,5-a]pyridine-2-carboxylic acid, 2-methyl triazolo[1,5-a]pyridin-3-ylamine, (3-aminopyrazolo[1,5-a]pyridin-7-yl)methanol, 2-(3-aminopyrazolo[1,5-a]pyrid-5-yl)ethanol, 2-(3-amino pyrazolo[1,5-a]pyrid-7-yl)ethanol, (3-aminopyrazolo[1,5-a]pyrid-2-yl)methanol, 3,6-diamino pyrazolo[1,5-a]pyridine, 3,4-diaminopyrazolo[1,5-a]pyridine, pyrazolo [1,5-a]pyridine-3,7-diamine, 7-morpholin-4-yl pyrazolo[1,5-a]pyridin-3-ylamine, pyrazolo[1,5-a]pyridine-3,5-diamine, 5-morpholin-4-yl pyrazolo[1,5-a]pyridin-3-ylamine, 2-[(3-aminopyrazolo[1,5-a]pyrid-5-yl)(2-hydroxyethyl)amino]ethanol, 2-[(3-amino pyrazolo[1,5-a]pyrid-7-yl)(2-hydroxyethyl)amino]ethanol, 3-aminopyrazolo[1,5-a]pyridin-5-ol, 3-amino pyrazolo[1,5-a]pyridin-4-ol, 3-aminopyrazolo[1,5-a]pyridin-6-ol, aminopyrazolo[1,5-a]pyridin-7-ol, 2-.alpha.-hydroxyethoxy-3-amino-pyrazolo[1,5-a]pyridine; 2-(4-dimethyl piperazine-1-yl)-3-amino-pyrazolo[1,5-a]pyridine; hydroxyethoxy aminopyrazole pyridine, and the salts thereof.

Oxidation bases that may be used in the present disclosure may be selected from 3-aminopyrazolo-[1,5-a]-pyridines and optionally substituted on carbon atom 2 by: (a) one (di)(C.sub.1-C.sub.6)(alkyl)amino group wherein said alkyl group can be substituted by at least one hydroxyl, amino, imidazolium group; (b) one heterocyclic alkyl group containing from 5 to 7 members chain, and from 1 to 3 heteroatoms, potentially cationic, potentially substituted by one or more (C.sub.1-C.sub.6)alkyl, such as di(C.sub.1-C.sub.4)alkyl pyridinium; or (c) one (C.sub.1-C.sub.6)alkoxy potentially substituted by one or more hydroxy groups such as .alpha.-hydroxy alkoxy, and the salts thereof.

The pyrimidine derivatives mentioned previously may be selected from 2,4,5,6-tetraaminopyrimidine, hydroxy-2,5,6-triaminopyrimidine, 2-hydroxy-4,5,6-triaminopyrimidine, 2,4-dihydroxy-5,6-diaminopyrimidine, 2,5,6-triaminopyrimidine and their salts and tautomeric forms, when a tautomeric equilibrium exists.

The pyrazole derivatives may be chosen from 4,5-diamino-1-methylpyrazole, 4,5-diamino-1-(.beta.-hydroxyethyl)pyrazole, 3,4-diamino pyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazine pyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(.beta. -hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethyl pyrazole, 4,5-diamino-3-hydroxymethyl-1-methylpyrazole, 4,5-diamino-3-hydroxymethyl-1-isopropyl pyrazole, 4,5-diamino-3-methyl-1-isopropyl pyrazole, 4-amino-5-(2′-aminoethyl)amino-1,3-dimethylpyrazole, 3,4,5-diamino pyrazole, 1-methyl-3,4,5-diamino pyrazole, 3,5-diamino-1-methyl-4-methylamino pyrazole, 3,5-diamino-4-(.beta.-hydroxyethyl)amino-1-methylpyrazole, and the salts thereof. In addition, 4,5-Diamino-1-(beta-methoxyethyl)pyrazole may also be used. According to some embodiments, 2,3-diamino dihydropyrazole pyrazolone dimethanesulfonate may also be used. Optionally, a 4,5-diaminopyrazole may be used, which may include, for example

4,5-diamino-1-(beta-hydroxyethyl)pyrazole and/or a salt thereof.

Pyrazole derivatives may be chosen from diamino-N,N-dihydropyrazole pyrazolones salts thereof, including: 2,3-diamino-6,7-dihydro-1H,5H-pyrazolo[1,2-a]pyrazol-1-one, 2-amino-3-ethylamino-6,7-dihydro-1H,5H-pyrazolo[1,2-a]pyrazol-1-one, 2-amino-3-isopropylamino-6,7-dihydro-1H,5H-pyrazolo[1,2-a]pyrazol-1-one, 2-amino-3-(pyrrolidin-1-yl)-6,7-dihydro-1H,5H-pyrazolo[1,2-a]pyrazol-1-on-e, 4,5-diamino-1,2-dimethyl-1,2-dihydropyrazol-3-one, 4,5-diamino-1,2-diethyl-1,2-dihydropyrazol-3-one, 4,5-diamino-1,2-di-(2-hydroxyethyl)-1,2-dihydropyrazol-3-one, 2-amino-3-(2-hydroxyethyl)amino-6,7-dihydro-1H,5H-pyrazolo[1,2-a]pyrazol-1-one, 2-amino-3-dimethylamino-6,7-dihydro-1H,5H-pyrazolo [1,2-a]pyrazol-1-one, 2,3-diamino-5,6,7,8-tetrahydro-1H,6H-pyridazino[1,2-a]pyrazol-1-one, 4-amino-1,2-diethyl-5-(pyrrolidin-1-yl)-1,2-dihydropyrazol-3-one, 4-amino-5-(3-dimethylaminopyrrolidin-1-yl)-1,2-diethyl-1,2-dihydropyrazol-3-one, and 2,3-diamino-6-hydroxy-6,7-dihydro-1H,5H-pyrazolo[1,2-a]pyrazol-1-one. For example, 2,3-Diamino-6,7-dihydro-1H,5H-pyrazolo[1,2-a]pyrazol-1-one and/or a salt thereof may be used.

According to some embodiments, 2,3-diamino dihydropyrazole pyrazolone dimethanesulfonate may be used. 4,5-Diamino-1-(beta-hydroxyethyl)pyrazole and/or 2,3-diamino-6,7-dihydro-1H,5H-pyrazolo[1,2-a]pyrazol-1-one and/or a salt thereof may be used as heterocyclic bases.

Chromophores may be constructed using three components: (1) 1,4-diaminobenzene (historically) or 2,5-diaminotoluene (currently), (2) a coupling agent, and (3) an oxidant. The process may be performed under basic conditions. The mechanism of oxidation dyes may involve three steps: 1) oxidation of 1,4-diaminobenzene derivative to the quinone state; 2) reaction of the diimine with a coupler compound; and 3) oxidation of the resulting compound to give the final chromophore. The coupling compound may be useful in adding a desired hydroxyl or amine pendant group.

Compositions according to the invention may optionally comprise one or more couplers advantageously chosen from those conventionally used in the preparation of chromophores. Among these couplers, meta-phenylenediamines, meta-aminophenols, meta-diphenols, naphthalene-based couplers and heterocyclic couplers, and also the salts thereof may be used.

Additionally, 2-methyl-5-aminophenol, 5-N-(.beta.-hydroxyethyl)amino-2-methylphenol, 3-aminophenol, 5-amino-6-chloro-o-cresol (3-amino-2-chloro-6-methylphenol), 1,3-dihydroxybenzene, 1,3-dihydroxy-2-methyl-benzene, 4-chloro-1,3-dihydroxybenzene, 2,4-diamino-1-(.beta.-hydroxyethoxy)-benzene (2,4 diaminophenoxyethanol HCL), 2-amino-4-(.beta.-hydroxyethylamino)-1-methoxybenzene (2-methyl-5-hydroxyethylaminophenol), 1,3-diaminobenzene, 1,3-bis(2,4-diamino phenoxy)propane, 3-ureido aniline, 3-ureido-1-dimethylaminobenzene, sesamol, 1-.beta.-hydroxyethyl amino-3,4-methylenedioxybenzene, .alpha.-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(.beta.-hydroxyethyl)amino-3,4-methylenedioxybenzene, 2,6-bis(.beta.-hydroxyethyl-amino)toluene, 6-hydroxyindoline, 2,6-dihydroxy-4-methylpyridine, 1-H-3-methyl pyrazol-5-one, 1-phenyl-3-methyl pyrazol-5-one, 2,6-dimethyl pyrazolo[1,5-b]-1,2,4-triazole, 2,6-dimethyl[3,2-c]-1,2,4-triazole, 4-amino-2-hydroxytoluene, 2-methylresorcinol, 4-chlororesorcinol, and 6-methylpyrazolo[1,5-a]benzimidazole, their salts, and mixtures thereof.

The salts of the oxidation bases and couplers that may be used in the context of the invention may be selected from the salts with an acid such as the hydrochlorides, hydrobromides, sulfates, citrates, succinates, tartrates, lactates, tosylates, benzenesulfonates, phosphates, and acetates.

Compositions according to the invention may optionally comprise one or more synthetic or natural direct dyes, chosen from anionic and nonionic species, e.g. cationic or nonionic species, either as sole dyes or in addition to the oxidation dye(s).

Direct dyes that may be utilized include azo direct dyes or dyes augmented with a hydroxyl group or amine group; (poly)methine dyes such as cyanins, hemicyanins and sterols; carbonyl dyes; azine dyes; nitro(hetero)aryl dyes; tri(hetero)aryl methane dyes; porphyrin dyes; phthalocyanine dyes, and natural direct dyes, alone or as mixtures.

Among the natural direct dyes that may be used directly or with a hydroxyl group or amine group added according to the invention, may include lawsone, juglone, alizarin, purpurin, carminic acid, kermesic acid, purpurogallin, protocatechualdehyde, indigo, isatin, curcumin, spinulosin, apigenin and orceins. Additionally, extracts or decoctions containing these natural dyes may be used.

Components of Use in a Polymeric Hair Colorant Composition

The polymer hair colorant of the present invention may be added to any auxiliary or additional component suitable for use in cosmetic compositions, and in particular, suitable for hair coloring. Such components may include, but are not limited to, cosmetically acceptable solvents, silicone compounds, thickening agents, rheology modifying agents such as acrylic polymers, anionic, cationic, nonionic, amphoteric or zwitterionic surfactants or mixtures thereof, anionic, cationic, nonionic, amphoteric or zwitterionic polymers or mixtures, film forming agents or polymers, humectants and moisturizing agents, fatty substances, emulsifying agents other than fatty substances, fillers, structuring agents, propellants, shine agents, conditioning agents, antioxidants or reducing agents, penetrants, sequestrants, fragrances, buffers, dispersants, conditioning agents, for instance volatile or non-volatile, modified or unmodified silicones, ceramides, preserving agents, opacifiers, sunscreen agents, and antistatic agents.

The polymeric hair colorant composition of the present disclosure may be in various forms, such as in the form of liquids, creams, liquid-gels, liquid-creams, gels, lotions or pastes.

The foregoing optional components may be present in amounts up to about 25%, such as about 1% to about 20%, or about 2% to about 10%, by weight of the composition, when present, although different amounts are also contemplated.

The polymeric hair colorant composition according to the disclosure may comprise a cosmetically acceptable solvent. The cosmetically acceptable solvent may be anhydrous.

Examples of organic solvents may include mono alcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol.

Other suitable examples of organic solvents may include ethylene glycol, propylene glycol, butylene glycol, hexylene glycol, propanediol, and glycerin. The organic solvents for use according to the present disclosure may be volatile or non-volatile compounds.

When present, the organic solvent may be employed according to the present disclosure in an amount ranging from about 0.1% to about 25% by weight, such as from about 1% to about 15% by weight, or such as from about 2% to about 8% by weight, or such as from about 3% to about 7% by weight, or such as about 4% to 6% by weight, based on the total weight of the polymeric hair colorant composition in which it is present. In one embodiment, the polymeric hair colorant composition may include 0.6 wt % of a carbomer (4% solution).

In various embodiments, the cosmetically acceptable solvent may be employed according to the present disclosure in an amount ranging from about 1% to about 60% by weight, or such as from about 5% to about 30% by weight, such as from about 5% to about 25% by weight, or such as from about 5% to about 20% by weight, based on the total weight of the polymeric hair colorant composition.

The polymeric hair colorant composition according to the disclosure may also comprise at least one nonionic surfactant. Nonionic surfactants having a hydrophilic-lipophilic balance of from 8 to 20, may be used in the present disclosure.

Nonionic surfactants useful in the compositions of the present disclosure may be selected from those disclosed in McCutcheon's “Detergents and Emulsifiers,” North American Edition (1986), published by Allured Publishing Corporation; and McCutcheon's “Functional Materials,” North American Edition (1992); both of which are incorporated by reference herein in their entirety.

Examples of nonionic surfactants useful herein may include, but are not limited to, alkoxylated derivatives of the following: fatty alcohols, alkyl phenols, fatty acids, fatty acid esters and fatty acid amides, wherein the alkyl chain is in the C.sub.12-C.sub.50 range, for example in the C.sub.16-C.sub.40 range, or in the C.sub.24 to C.sub.40 range, and having from about 1 to about 110 alkoxy groups. The alkoxy groups may be selected from C.sub.2-C.sub.6 oxides and their mixtures, with ethylene oxide, propylene oxide, and their mixtures being useful examples. The alkyl chain may be linear, branched, saturated, or unsaturated. Of these alkoxylated non-ionic surfactants, the alkoxylated alcohols may be chosen, such as ethoxylated alcohols and propoxylated alcohols. The alkoxylated alcohols may be used alone or in mixtures thereof. The alkoxylated alcohols may also be used in mixtures with those alkoxylated materials disclosed herein-above.

Ethoxylated fatty alcohols to be used may include laureth-3 (a lauryl ethoxylate having an average degree of ethoxylation of 3), laureth-23 (a lauryl ethoxylate having an average degree of ethoxylation of 23), ceteth-10 (a cetyl alcohol ethoxylate having an average degree of ethoxylation of 10) steareth-10 (a stearyl alcohol ethoxylate having an average degree of ethoxylation of 10), and steareth-2 (a stearyl alcohol ethoxylate having an average degree of ethoxylation of 2), steareth-20 (a stearyl alcohol ethoxylate having an average degree of ethoxylation of 20), steareth-100 (a stearyl alcohol ethoxylate having an average degree of ethoxylation of 100), beheneth-5 (a behenyl alcohol ethoxylate having an average degree of ethoxylation of 5), beheneth-10 (a behenyl alcohol ethoxylate having an average degree of ethoxylation of 10), and other derivatives and mixtures of the preceding.

Also available commercially are Brij® nonionic surfactants from Croda, Inc., Edison, N.J. Brij® may be the condensation products of aliphatic alcohols with from about 1 to about 54 moles of ethylene oxide, the alkyl chain of the alcohol being typically a linear chain and having from about 8 to about 22 carbon atoms, for example, Brij® 72 (i.e., Steareth-2) and Brij® 76 (i.e., Steareth-10).

Also useful herein as nonionic surfactants are alkyl glycosides, which are the condensation products of long chain alcohols, e.g. C.sub.8-C.sub.30 alcohols, with sugar or starch polymers. These compounds may be represented by the formula (S)n-O—R wherein S is a sugar moiety such as glucose, fructose, mannose, galactose, and the like; “n” is an integer of from about 1 to about 1000, and R is a C.sub.8-C.sub.30 alkyl group. Examples of long chain alcohols from which the alkyl group can be derived may include decyl alcohol, cetyl alcohol, stearyl alcohol, lauryl alcohol, myristyl alcohol, oleyl alcohol, and the like. Examples of these surfactants may include alkyl polyglucosides wherein S is a glucose moiety, R is a C.sub.8-C.sub.20 alkyl group, and “n” is an integer of from about 1 to about 9. Commercially available examples of these surfactants may include decyl polyglucoside (available as APG® 325 CS) and lauryl polyglucoside (available as APG® 600 CS and 625 CS), all the above-identified polyglucosides APG® are available from BASF Corp. Sucrose ester surfactants such as sucrose cocoate and sucrose laurate may also be used in some embodiments.

Other nonionic surfactants suitable for use according to the present disclosure are glyceryl esters and polyglyceryl esters, including but not limited to, glyceryl monoesters, such as glyceryl monoesters of C.sub.16-C.sub.22 saturated, unsaturated and branched chain fatty acids such as glyceryl oleate, glyceryl monostearate, glyceryl monoisostearate, glyceryl monopalmitate, glyceryl mono behenate, and mixtures thereof, and polyglyceryl esters of C.sub.16-C.sub.22 saturated, unsaturated and branched chain fatty acids, such as polyglyceryl-4 isostearate, polyglyceryl-3 oleate, polyglyceryl-2 sesquioleate, triglyceryl diisostearate, diglyceryl monooleate, tetraglyceryl monooleate, and mixtures thereof.

Also useful herein as nonionic surfactants are sorbitan esters, e.g. sorbitan esters of C.sub.16-C.sub.22 saturated, unsaturated and branched chain fatty acids. Because of the manner in which they are typically manufactured, these sorbitan esters usually comprise mixtures of mono-, di-, tri-, etc. esters. Representative examples of suitable sorbitan esters include sorbitan monooleate (e.g., SPAN® 80), sorbitan sesquioleate (e.g., Arlacel® 83 from Croda, Inc., Edison, N.J.), sorbitan monoisostearate (e.g., CRILL® 6 from Croda, Inc., Edison, N.J.), sorbitan stearates (e.g., SPAN® 60), sorbitan trioleate (e.g., SPAN® 85), sorbitan tristearate (e.g., SPAN® 65), sorbitan dipalmitates (e.g., SPAN® 40), and sorbitan isostearate. Sorbitan monoisostearate and sorbitan sesquioleate may be used as emulsifiers.

Also suitable for use herein are alkoxylated derivatives of glyceryl esters, sorbitan esters, and alkyl polyglycosides, wherein the alkoxy groups is selected from C.sub.2-C.sub.6 oxides and their mixtures, such as ethoxylated or propoxylated derivatives of these materials. Non limiting examples of commercially available ethoxylated materials may include TWEEN® (ethoxylated sorbitan mono-, di- and/or tri-esters of C.sub.12 to C.sub.18 fatty acids with an average degree of ethoxylation of from about 2 to about 20).

According to some embodiments TWEEN®-21 (polyoxyethylene (4) sorbitan monolaurate) may be used.

Nonionic surfactants may be those formed from a fatty alcohol, a fatty acid, or a glyceride with a C.sub.4 to C.sub.36 carbon chain, such as a C.sub.12 to C.sub.18 carbon chain, or a C.sub.16 to C.sub.18 carbon chain, derivatized to yield an HLB of at least 8. HLB may be understood to mean the balance between the size and strength of the hydrophilic group and the size and strength of the lipophilic group of the surfactant. Such derivatives may be polymers such as ethoxylates, propoxylates, polyglucosides, polyglycerins, polylactates, polyglycolates, polysorbates, and others that would be apparent to one of ordinary skill in the art. Such derivatives may also be mixed polymers of the above, such as ethoxylate/propoxylate species, where the total HLB is optionally greater than or equal to 8.

In various embodiments, the nonionic surfactants contain ethoxylate in a molar content of from 10-25, such as from 10-20 moles.

When present in the polymeric hair colorant composition, the nonionic surfactant may comprise an amount of from about 0.1% to about 30% by weight, such as from about 0.5% to 20% by weight, from about 1% to about 12% by weight, or from about 5% to about 9% by weight, based on the total weight of the polymeric hair colorant composition.

The polymeric hair colorant composition according to the disclosure may comprise at least one fatty substance. Fatty substances may have in their structure a chain of at least two siloxane groups or at least one hydrocarbon chain having at least 6 carbon atoms. Moreover, fatty substances may be soluble in organic solvents in the same conditions of temperature and pressure, for example in chloroform, ethanol, benzene or decamethylcyclopentasiloxane.

Fatty substances may be chosen from alkanes, fatty alcohols, esters of fatty acid, esters of fatty alcohol, oils such as mineral, vegetable, animal and synthetic non-silicone oils, non-silicone waxes and silicones.

In some embodiments, the alcohols and esters may have at least one linear or branched, saturated or unsaturated hydrocarbon group, comprising 6 to 30 carbon atoms, optionally substituted, for example, with at least one hydroxyl group. If they are unsaturated, these compounds may have one to three, conjugated or unconjugated, carbon-carbon double bonds.

With regard to the alkanes, in some embodiments, these alkanes may have from 6 to 16 carbon atoms and are linear or branched, optionally cyclic. As examples, alkanes can be chosen from hexane, and dodecane, and isoparaffins such as isohexadecane, isododecane, and isodecane.

In one embodiment, non-silicone oils according to the disclosure may be used, which may include but are not limited to hydrocarbon oils of animal origin, such as perhydro squalene; hydrocarbon oils of vegetable origin, such as liquid triglycerides of fatty acids having from 6 to 30 carbon atoms such as oleic acid, triglycerides of heptanoic or octanoic acids, or for example sunflower oil, maize oil, soya oil, cucurbit oil, grapeseed oil, sesame oil, hazelnut oil, apricot oil, macadamia oil, arara oil, sunflower oil, castor oil, avocado oil, argan oil, flaxseed oil, pomegranate oil, rosehip oil, triglycerides of caprylic/capric acids such as those sold by the company Stearineries Dubois or those sold under the names MIGLYOL® 810, 812 and 818 by the company Dynamit Nobel, jojoba oil, shea butter oil; hydrocarbons with more than 16 carbon atoms, linear or branched, of mineral or synthetic origin, such as paraffin oils, petroleum jelly, liquid paraffin, polydecenes, hydrogenated polyisobutene such as Parleam® fluorinated, partially hydrocarbon oils; as fluorinated oils, non-limiting examples include perfluoro methyl cyclopentane and perfluoro-1,3-dimethylcyclohexane, sold under the names “FLUTEC® PC1” and “FLUTEC® PC3” by the company F2 Chemicals Ltd.; perfluoro-1,2-dimethylcyclobutane; perfluoroalkanes such as dodecafluoropentane and tetradecafluorohexane, sold under the names “PF 5050e” and “PF 5060e” by the 3M Company, or bromo perfluorooctyl sold under the name “FORALKYL®” by the company Atochem; nonafluoro-methoxybutane and nonafluoroethoxyisobutane; derivatives of perfluoro morpholine, such as 4-trifluoromethyl perfluoromorpholine sold under the name “PF 5052e” by the 3M Company.

The fatty alcohols usable as fatty substances according to the disclosure may include, but are not limited to, non-alkoxylated, saturated or unsaturated, linear or branched, and have from 6 to 30 carbon atoms and more particularly from 8 to 30 carbon atoms. For example, cetyl alcohol, stearyl alcohol and their mixture (cetearyl alcohol), octyldodecanol, 2-butyloctanol, 2-hexyldecanol, 2-undecyl pentadecanol, oleic alcohol or linoleic alcohol may be chosen.

Non-silicone wax or waxes may be used in the composition of the disclosure and may be selected from paraffin wax, carnauba wax, candelilla wax, Alfa wax, ozokerite, vegetable waxes such as olive wax, rice wax, hydrogenated jojoba wax or absolute waxes of flowers such as the essential wax of blackcurrant flower sold by the company BERTIN (France), animal waxes such as beeswaxes, or modified beeswaxes (cerabellina); other waxes or waxy raw materials usable according to the disclosure are, for example, marine waxes such as that sold by the company SOPHIM under reference M82, waxes of polyethylene or of polyolefins, or any combinations thereof.

Fatty acid esters of the current disclosure may include esters of saturated or unsaturated, linear or branched C.sub.1-C.sub.26 aliphatic mono- or polyacids and of saturated or unsaturated, linear or branched C.sub.1-C.sub.26 aliphatic mono- or polyalcohols, the total number of carbons of the esters being, for example, greater than or equal to 10.

Among the monoesters, non-limiting example may include dihydroabietyl behenate; octyldodecyl behenate; isocetyl behenate; cetyl lactate; C.sub.12-C.sub.15 alkyl lactate; isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methyl acetyl ricinoleate; myristyl stearate; octyl isononanoate; 2-ethylhexyl isononate; octyl palmitate; octyl pelargonate; octyl stearate; octyldodecyl erucate; oleyl erucate; ethyl and isopropyl palmitates, ethyl-2-hexyl palmitate, 2-octyldodecyl palmitate, alkyl myristates such as isopropyl, butyl, cetyl, 2-octyldodecyl, mirystyl, stearyl myristate, hexyl stearate, butyl stearate, isobutyl stearate; dioctyl malate, hexyl laurate, and 2-hexyldecyl laurate, their salts and combinations thereof.

Further non-limiting examples of esters may include esters of C.sub.4-C.sub.22 di- or tricarboxylic acids and of C.sub.1-C.sub.22 alcohols and the esters of mono-, di- or tricarboxylic acids and of C.sub.2-C.sub.26 di-, tri-, tetra- or pentahydroxy alcohols.

In further embodiments, non-limiting examples of esters may be selected from diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate, tridecyl erucate; triisopropyl citrate; triisostearyl citrate; glyceryl trilactate; glyceryl trioctanoate; trioctyldodecyl citrate; trioleyl citrate, propylene glycol dioctanoate; neopentyl glycol diheptanoate; diethylene glycol diisanonate; and polyethylene glycol distearate.

Among the esters mentioned above, exemplary esters include ethyl, isopropyl, myristyl, cetyl, stearyl palmitates, ethyl-2-hexyl palmitate, 2-octyldodecyl palmitate, alkyl myristates such as isopropyl, butyl, cetyl, 2-octyldodecyl myristate, hexyl stearate, butyl stearate, isobutyl stearate; dioctyl malate, hexyl laurate, 2-hexyldecyl laurate and isononyl isononanoate, cetyl octanoate, their salts and mixtures thereof.

In one embodiment, the composition may also comprise, as fatty ester, esters and di-esters of sugars of C.sub.6-C.sub.30, such as C.sub.12-C.sub.22 fatty acids. “Sugar” as used in the disclosure may be understood to comprise oxygen-containing hydrocarbon compounds that possess several alcohol functions, with or without aldehyde or ketone functions, and having at least 4 carbon atoms. These sugars may be monosaccharides, oligosaccharides, or polysaccharides.

As suitable sugars, non-limiting examples may include sucrose, glucose, galactose, ribose, fucose, maltose, fructose, mannose, arabinose, xylose, lactose, and their derivatives, for example alkylated, such as methylated derivatives such as methylglucose.

The esters of sugars and of fatty acids may, for example, be chosen from the esters or mixtures of esters of sugars described previously and of linear or branched, saturated or unsaturated C.sub.6-C.sub.30, such as C.sub.12-C.sub.22 fatty acids. If they are unsaturated, these compounds may have one to three, conjugated or unconjugated, carbon-carbon double bonds.

The esters according to at least one embodiment may also be chosen from mono-, di-, tri- and tetra-esters, polyesters and mixtures thereof. These esters can be for example oleate, laurate, palmitate, myristate, behenate, cocoate, stearate, linoleate, linolenate, caprate, arachidonates, or mixtures thereof such as the oleo palmitate, oleo-stearate, palmitostearate mixed esters.

For example, the mono- and di-esters may be used and may be selected from mono- or di-oleate, stearate, behenate, oleo palmitate, linoleate, linolenate, oleostearate, of sucrose, of glucose or of methylglucose. Non-limiting examples may be of the product sold under the name GLUCATE® DO by the company Amerchol, which is a dioleate of methylglucose.

Some embodiments may include esters or mixtures of esters of sugar of fatty acid selected from the products sold under the names Crodesta™ F160, F140, F110, F90, F70, SL40 by the company Croda, Inc., Edison, N.J., denoting respectively the palmito-stearates of sucrose formed from 73% of monoester and 27% of di- and tri-ester, from 61% of monoester and 39% of di-, tri-, and tetra-ester, from 52% of monoester and 48% of di-, tri-, and tetra-ester, from 45% of monoester and 55% of di-, tri-, and tetra-ester, from 39% of monoester and 61% of di-, tri-, and tetra-ester, and the monolaurate of sucrose; the products sold under the name Ryoto Sugar Esters for example with the reference B370 and corresponding to the behenate of sucrose formed from 20% of monoester and 80% of di-triester-polyester; sucrose mono-di-palmito-stearate marketed by the company Evonik under the name TEGOSOFT® PSE.

The silicones usable in the composition of the present disclosure include but are not limited to volatile or non-volatile, cyclic, linear or branched silicones, modified or not with organic groups.

The silicones usable according to the disclosure may be in the form of oils, waxes, resins or gums. In some embodiments, a mixture C30-45 alkyl dimethyl silyl polypropylsilsesquioxane, available as SW-8005 C30 resin wax available from DOW CORNING®, and paraffin, may be used.

In some embodiments, the silicone is selected from polydialkylsiloxanes, such as the polydimethylsiloxanes (PDMS), and the organomodified polysiloxanes having at least one functional group selected from the poly(alkoxylated) groups and the alkoxy groups.

Organopolysiloxanes may be defined in more detail in the work of Walter NOLL “Chemistry and Technology of Silicones” (1968), Academic Press. Such compositions may be volatile or non-volatile.

In some embodiments, silicones may be volatile. Such compounds may be chosen from those with a boiling point between 60.degree. C. and 260.degree. C. Further examples may include those chosen from cyclic polydialkylsiloxanes having from 3 to 7 silicon atoms, more preferably from 4 to 5 silicon atoms. Non-limiting examples may include octamethylcyclotetrasiloxane marketed under the name VOLATILE SILICONE® 7207 by UNION CARBIDE or SILBIONE® 70045 V2 by RHODIA, decamethylcyclopentasiloxane marketed under the name VOLATILE SILICONE® 7158 by UNION CARBIDE, and SILBIONE® 70045 V5 by RHODIA, and mixtures thereof.

Further, non-limiting may also be cyclocopolymers of the dimethylsiloxanes/methylalkylsiloxane type, such as SILICONE VOLATILE® FZ 3109 marketed by the company UNION CARBIDE.

Some embodiments may comprise mixtures of cyclic polydialkylsiloxanes with organic compounds derived from silicon, such as the mixture of octamethylcyclotetrasiloxane and tetratrimethylsily pentaerythritol (50/50) and the mixture of octamethylcyclotetrasiloxane and oxy-1,1′-(hexa-2,2,2′,2′,3,3′-trimethylsilyloxy) bis-neopentane.

In some embodiments, suitable volatile silicones may include the linear volatile polydialkylsiloxanes having 2 to 9 silicon atoms and with a viscosity less than or equal to 5.times.10.sup.-6 m.sup.2/s at 25.degree. C. One example may include decamethyltetrasiloxane, marketed under the name “Toray SH 200 Fluid” by the company DOW CORNING®. Silicones included in this class may also be described in the article published in Cosmetics and Toiletries, Vol. 91, January 76, p. 27-32-TODD BYERS “Volatile Silicone fluids for cosmetics”.

Further non-limiting examples may be made of non-volatile polydialkylsiloxanes, gums and resins of polydialkylsiloxanes, polyorganosiloxanes modified with the aforementioned organofunctional groups, and mixtures thereof.

These silicones are, for example, selected from the polydialkylsiloxanes, such as the polydimethylsiloxanes with trimethylsilyl end groups.

Embodiments comprising polydialkylsiloxanesmay include the following commercial products SILBIONE® oils of series 47 and 70 047 or MIRASIL® oils marketed by RHODIA, for example the oil 70 047 V 500 000; oils of the MIRASIL® series marketed by the company RHODIA; oils of the 200 series from the company DOW CORNING such as DC200; VISCASIL® oils from GENERAL ELECTRIC and certain oils of the SF series (SF 96, SF 18) from GENERAL ELECTRIC.

Non-limiting examples may also include polydimethylsiloxanes with dimethylsilanol end groups known under the name of dimethiconol (CTFA), such as oils of the 48 series from the company RHODIA.

Polydialkylsiloxanes may also include products marketed under the names “ABIL WAX® 9800 and 9801” by the company Evonik, which may include polydialkyl (C.sub.1-C.sub.20) siloxanes.

In some embodiments, silicone gums may be used according to the present disclosure. Silicone gums may include, for example, polydialkylsiloxanes, such as polydimethylsiloxanes with high number-average molecular weights between 200,000 and 1,000,000 used alone or mixed in a solvent. The solvent may be chosen from the volatile silicones, the polydimethylsiloxane (PDMS) oils, the polyphenylmethylsiloxane (PPMS) oils, the isoparaffins, the polyisobutylenes, methylene chloride, pentane, dodecane, tridecane and mixtures thereof.

Some embodiments may include mixtures such as: mixtures formed from a chain end hydroxylated polydimethylsiloxane, or dimethiconol (CTFA) and a cyclic polydimethylsiloxane also called cyclomethicone (CTFA), such as the product Q2 1401 marketed by the company DOW CORNING; mixtures of a polydimethylsiloxane gum and a cyclic silicone such as the product SF 1214 Silicone Fluid from the company GENERAL ELECTRIC, said product being a gum SF 30 corresponding to a dimethicone, having a number-average molecular weight of 500,000, dissolved in the oil SF 1202 Silicone Fluid corresponding to decamethylcyclopentasiloxane; mixtures of two PDMS of different viscosities, for example, of a PDMS gum and a PDMS oil, such as the product SF 1236 from the company GENERAL ELECTRIC. The product SF 1236 is a mixture of a gum SE 30 as defined above having a viscosity of 20 m.sup.2/s and an oil SF 96 with a viscosity of 5.times.10.sup.-6 m.sup.2/s. SF 1236, for example, has 15% of gum SE 30 and 85% of oil SF 96.

The organopolysiloxane resins of some embodiments according to the disclosure may include, but are not limited to, crosslinked siloxane systems containing the units: R.sub.2SiO.sub.2/2, R.sub.3SiO.sub.1/2, RSiO.sub.3/2 and SiO.sub.4/2, in which R represents an alkyl having 1 to 16 carbon atoms. For example, R may denote a C.sub.1-C.sub.4 lower alkyl group such as methyl.

Among these resins, non-limiting examples may include the product marketed under the name “DOW CORNING 593” or those marketed under the names “SILICONE FLUID SS 4230 and SS 4267” by the company GENERAL ELECTRIC, which are silicones of dimethyl/trimethyl siloxane structure.

Non-limiting examples may also include resins of the trimethylsiloxysilicate type, such as those marketed under the names X22-4914, X21-5034 and X21-5037 by the company SHIN-ETSU.

Organomodified silicones of some embodiments may include, but are not limited to, silicones as defined previously, having in their structure at least one organofunctional group fixed by a hydrocarbon group.

In addition to the silicones described above, the organomodified silicones may be polydiaryl siloxanes, such as polydiphenylsiloxanes, and polyalkyl-arylsiloxanes functionalized by the aforementioned organofunctional groups.

The polyalkarylsiloxanes are, for example, selected from the group of polydimethyl/methylphenylsiloxanes, the polydimethyl/diphenylsiloxanes, linear and/or branched, with viscosity ranging from 1.times.10.sup.-5 to 5.times.10.sup.2 m.sup.2/s at 25.degree. C.

Further polyalkarylsiloxanes examples may include the products marketed under the following names: the SILBIONE® oils of series 70 641 from RHODIA; the oils of the series RHODORSIL® 70 633 and 763 from RHODIA; the oil DOW CORNING 556 COSMETIC GRADE FLUID from DOW CORNING; the silicones of the PK series from BAYER such as the product PK20; the silicones of the series PN, PH from BAYER such as the products PN1000 and PH1000; certain oils of the SF series from GENERAL ELECTRIC such as SF 1023, SF 1154, SF 1250, SF 1265.

Some embodiments may comprise organo modified silicones, which may be made of the polyorganosiloxanes having: polyoxyethylene and/or polyoxypropylene groups optionally with C.sub.6-C.sub.24 alkyl groups such as the products called dimethicone copolyol marketed by the company DOW CORNING under the name DC 1248 or the oils SILWET® L 722, L 7500, L 77, L 711 from the company UNION CARBIDE and the alkyl (C.sub.12)-methicone copolyol marketed by the company DOW CORNING under the name Q2 5200; substituted or unsubstituted amine groups such as the products marketed under the name GP 4 Silicone Fluid and GP 7100 by the company Genesee Polymers, or the products marketed under the names Q2 8220 and DOW CORNING 929 or 939 by the company DOW CORNING. The substituted amine groups are, for example, C.sub.1-C.sub.4 aminoalkyl groups; alkoxylated groups, such as the product marketed under the name “SILICONE COPOLYMER F-755” by SWS SILICONES and ABIL WAX® 2428, 2434 and 2440 by the company Evonik.

Embodiments comprising a fatty substance may select compounds that are liquid or pasty at room temperature and at atmospheric pressure. The fatty substance may be a compound that is liquid at a temperature of 25.degree. C. and at atmospheric pressure.

The fatty substance may be chosen from alkanes, fatty alcohols, esters of fatty acid, esters of fatty alcohol, hydrocarbons, silicones, non-silicone oils, and non-silicone waxes.

The non-silicone oils may be selected from mineral, vegetable and synthetic oils.

According to at least one embodiment, the fatty substance may be selected from liquid paraffin, polydecenes, liquid esters of fatty acids and of fatty alcohols, and mixtures thereof. In some embodiments, the fatty substance may be alkanes, hydrocarbons, and silicones.

In some embodiments, the liquid fatty substances are advantageously chosen from C.sub.6-C.sub.16 alkanes, non-silicone oils of plant, mineral or synthetic origin, liquid fatty alcohols, liquid fatty acids and liquid esters of a fatty acid and/or of a fatty alcohol, or mixtures thereof. In various embodiments, the liquid fatty substance may be selected from liquid petroleum jelly, C.sub.6-C.sub.16 alkanes, polydecenes, liquid esters of a fatty acid and/or of a fatty alcohol, and liquid fatty alcohols, or mixtures thereof.

One example of a liquid fatty substance for use according to the present disclosure may include mineral oil which may be commercially available from the supplier Sonneborn under the trade name Kaydol® Heavy White Mineral Oil or from the supplier Exxonmobil Chemical under the trade name Primol® 352 or from Sonneborn under the trade name Blandol, or from Armedsa under the trade name Aemoil M-302CG or from Exxonmobil Chemical under the tradename Marcol 82.

According to some embodiments, a polymeric hair colorant composition may include mineral oil and cetearyl alcohol as fatty substances. For example, the polymeric hair colorant composition may include from about 3 to about 7 wt % mineral oil, such as about 4 to about 6 wt %, or about 5 wt %. The polymeric hair colorant composition may include from about 3 to about 7 wt % cetearyl alcohol, such as about 4 to about 6 wt %, or about 5 wt %. In some embodiments, the polymeric hair colorant composition may include approximately equal amounts of mineral oil and cetearyl alcohol.

In some embodiments, a fatty substance may have a viscosity of about 50 mm.sup.2/s or less at 40.degree. C. (kinematic viscosity as measured by the ASTM D 445 method in units of mm.sup.2/s at 40.degree. C.). In other embodiments, the fatty substance may have a viscosity of greater than about 50 mm2/s at 40.degree. C. and may be chosen from oils such as mineral oil (kinematic viscosity as measured by the ASTM D 445 method in units of mm2/s at 40.degree. C.).

The polymeric hair colorant composition may include at least one rheology modifier, for example, an acrylic polymer. When present, the acrylic polymer may, be selected from crosslinked copolymers of (meth)acrylic acid and/or (C1-C6)alkyl esters and from acrylic associative polymers.

The expression “acrylic polymer” is understood, for the purposes of the present disclosure, to comprise a polymer that results from the polymerization of one or more monomers.

The acrylic polymer of the present disclosure may also belong to a group of compounds known as acrylic thickening polymers.

The expression “thickening polymer” is understood, for the purposes of the present disclosure, to comprise a polymer having, in solution or in dispersion containing 1 percent by weight of active material in water or in ethanol at 25.degree. C., a viscosity greater than 0.2 poise ata shear rate of 1 s-1.

As used herein, the term “(meth)acrylic” acid and “(meth)acrylate” are understood to include the corresponding methyl derivatives of acrylic acid and the corresponding alkyl acrylate. For example, “(meth)acrylic)” acid may refer to acrylic acid and/or methacrylic acid, and “(meth)acrylate” may refer to alkyl acrylate and/or alkyl methacrylate.

In certain embodiments, the acrylic polymer of the present disclosure may be selected from crosslinked copolymers of methacrylic acid and of a C1-C6 alkyl ester wherein the C1-C6 alkyl ester is a C1-C6 alkyl acrylate.

Methacrylic acid may be present in amounts ranging from 20 percent to 80 percent by weight, more particularly from 25 percent to 70 percent by weight, such as from 35 percent to 65 percent by weight, relative to the total weight of the copolymer.

The alkyl acrylate may be present in amounts ranging from 15 percent to 80 percent by weight, such as from 25 percent to 75 percent by weight or from 35 percent to 65 percent by weight relative to the total weight of the copolymer. It may be selected from methyl acrylate, ethyl acrylate and butyl acrylate and more particularly ethyl acrylate.

In one embodiment, the copolymer of the present invention may be partially or totally/substantially crosslinked with at least one standard poly ethylenically unsaturated crosslinking agent. For example, polyalkenyl ethers of sucrose or of polyols, diallyl phthalates, divinylbenzene, allyl (meth)acrylate, ethylene glycol di(meth)acrylate, methylenebisacrylamide, trimethylolpropane tri(meth)acrylate, diallyl itaconate, diallyl fumarate, diallyl maleate, zinc (meth)acrylate, and castor oil or polyol derivatives manufactured from unsaturated carboxylic acids. The content of crosslinking agent may range from 0.01 percent to 5 percent by weight, such as from 0.03 percent to 3 percent by weight or from 0.05 percent to 1 percent by weight, relative to the total weight of the copolymer.

In certain embodiments, the crosslinked copolymer of methacrylic acid and of a C1-C6 alkyl acrylate may be slightly cross-linked. As used herein, the term “slightly crosslinked” may refer to a partially crosslinked three-dimensional polymeric network.

In some embodiments, the crosslinked copolymer of methacrylic acid and of a C1-C6 alkyl acrylate is alkali-swellable. As used herein, the term “alkali-swellable” as it pertains to the acrylic polymer of the present disclosure may refer to a polymer that when introduced to a solution, imparts little or no viscosity, but upon adjusting the pH to mildly acidic, neutral, or mildly basic conditions, a measurable increase in viscosity is observed, i.e., adding an alkali or neutralizing agent to a solution containing an alkali swellable polymer results in the development of viscosity.

The term “alkali-swellable” as used herein may also refer to the expansion of the polymer molecules upon neutralization as a result of charge repulsion of the anionic carboxylate groups of the polymer.

In some embodiments, an acrylic polymer of the present disclosure may be selected from a crosslinked (meth)acrylic acid/ethyl acrylate copolymer, a cross-linked anionic acrylate polymer, and mixtures thereof.

In certain embodiments, the crosslinked (meth)acrylic acid/ethyl acrylate copolymer may comprise a crosslinked methacrylic acid/ethyl acrylate copolymer, an example of which is a slightly cross-linked, alkali-swellable acrylate polymer known by the INCI name acrylates copolymer and commercially available from the supplier Lubrizol, under the trade name Carbopol® This copolymer belongs to a class of synthetic rheology modifiers that include carboxyl functional alkali-swellable and alkali-soluble thickeners (ASTs). These thickener polymers may be prepared from the free-radical polymerization of acrylic acid alone or in combination with other ethylenically unsaturated monomers. The polymers can be synthesized by solvent/precipitation as well as emulsion polymerization techniques.

In other certain embodiments, the acrylic polymer of the present disclosure may include a cross-linked anionic acrylate polymer. The cross-linked anionic acrylate polymer may be contained in a non-aqueous dispersion comprising about 32% by weight of total solids. Acrylates Crosspolymer-4 may also be described as a copolymer of acrylic acid, methacrylic acid or one of its simple esters, crosslinked with trimethylolpropane triacrylate.

In certain embodiments, the acrylic polymer of the present disclosure may be selected from acrylic associative polymers, also known as acrylic associative thickeners. The expression “associative thickener” may be understood to include an amphiphilic thickener comprising both hydrophilic units and hydrophobic units, in particular comprising at least one C8-C30 fatty chain and at least one hydrophilic unit.

Acrylic associative thickeners that may be used according to the invention may include acrylic associative polymers selected from: (i) nonionic amphiphilic polymers comprising at least one fatty chain and at least one hydrophilic unit; (ii) anionic amphiphilic polymers comprising at least one hydrophilic unit and at least one fatty-chain unit; (iii) cationic amphiphilic polymers comprising at least one hydrophilic unit and at least one fatty-chain unit; and (iv) amphoteric amphiphilic polymers comprising at least one hydrophilic unit and at least one fatty-chain unit; the fatty chains containing from 10 to 30 carbon atoms.

Acrylic associative polymers may be selected from acrylic anionic amphiphilic polymers such as those comprising at least one hydrophilic unit of unsaturated olefinic carboxylic acid type, and at least one hydrophobic unit of (C10-C30) alkyl ester of an unsaturated carboxylic acid. The anionic amphiphilic polymers of the present disclosure may denote polymers formed from a mixture of monomers.

Thickening agents and rheology modifying polymers other than acrylic polymers may be added. These may include, for example, polymeric thickeners and/or non-polymeric thickeners. The polymeric thickener can be chosen from ionic or non-ionic, associative or non-associative polymers. Exemplary polymeric thickeners may include various native gums. Representative non-polymeric thickening agents may include oxyethylenated molecules and especially ethoxylated alkyl or acyl derivatives of polyols. These polymers can be modified physically or chemically.

When present, the thickening agent may be employed in the compositions of the present disclosure in an amount of from greater than 0% to about 15% by weight, such as from about 0.1% to about 10% by weight, or from about 1% to about 5% by weight, based on the total weight of the polymeric hair colorant composition.

The compositions according to the present disclosure may also comprise at least one cationic polymer. The cationic polymer may be chosen from cationic associative polymers comprising a pendent or terminal hydrophobic chain, for example of alkyl or alkenyl type, containing from 10 to 30 carbon atoms.

A cationic polymer of one embodiment may be selected from homopolymers and copolymers derived from acrylic or methacrylic esters or amides, examples of which may include: copolymers of acrylamide and of dimethylaminoethyl acrylate quaternized with dimethyl sulfate or with a dimethyl halide, such as the product sold under the name HERCOFLOC by the company Hercules; the copolymers of acrylamide and of methacryloyloxyethyl trimethylammonium chloride described, for example, in EP 80 976 and sold under the name BINA QUAT P 100 by the company Ciba Geigy; the copolymer of acrylamide and of methacryloyloxyethyl trimethyl ammonium methosulfate sold under the name RETEN by the company Hercules; quaternized or non-quaternized vinylpyrrolidone/dialkylaminoalkyl acrylate or acrylate copolymers, such as the products sold under the name GAFQUAT by the company ISP, for instance GAFQUAT 734 or GAFQUAT 755, or alternatively the products known as COPOLYMER 845, 958 and 937, dimethylaminoethyl acrylate/vinylcaprolactam/vinylpyrrolidone terpolymers, such as the product sold under the name GAFFIX VC 713 by the company ISP; vinylpyrrolidone/methacrylamido propyl dimethylamine copolymers sold, for example, under the name STYLEZE CC 10 by ISP; quaternized vinylpyrrolidone/dimethylaminopropylmethacrylamide copolymers such as the product sold under the name GAFQUAT HS 100 by the company ISP, and crosslinked polymers of methacryloyloxy(C.sub.1-C.sub.4)alkyl tri(C.sub.1-C.sub.4)alkylammonium salts such as the polymers obtained by homopolymerization of dimethylaminoethyl acrylate quaternized with methyl chloride, or by copolymerization of acrylamide with dimethylaminoethyl acrylate quaternized with methyl chloride, the homo- or copolymerization being followed by crosslinking with a compound containing olefinic unsaturation, such as methylenebisacrylamide.

In some embodiments, cationic polymers may include, but are not limited to, polyquaternium 4, polyquaternium 6, polyquaternium 7, polyquaternium 10, polyquaternium 11, polyquaternium 16, polyquaternium 22, polyquaternium 28, polyquaternium 32, polyquaternium-46, polyquaternium-51, polyquaternium-52, polyquaternium-53, polyquaternium-54, polyquaternium-55, polyquaternium-56, polyquaternium-57, polyquaternium-58, polyquaternium-59, polyquaternium-60, polyquaternium-63, polyquaternium-64, polyquaternium-65, polyquaternium-66, polyquaternium-67, polyquaternium-70, polyquaternium-73, polyquaternium-74, polyquaternium-75, polyquaternium-76, polyquaternium-77, polyquaternium-78, polyquaternium-79, polyquaternium-80, polyquaternium-81, polyquaternium-82, polyquaternium-84, polyquaternium-85, polyquaternium-86, polyquaternium-87, polyquaternium-90, polyquaternium-91, polyquaternium-92, polyquaternium-94, and guar hydroxypropyltrimonium chloride.

Cationic polymers may further include POLYMER JR-125, POLYMER JR-400, Polymer JR-30M hydroxyethyl cellulosic polymers (polyquaternium 10) available from AMERCHOL; JAGUAR C® 13-S, guar hydroxypropyltrimonium chloride, available from Rhodia; and MERQUAT® 100 and 280, a dimethyl dialkyl ammonium chloride (polyquaternium 6) available from Nalco.

The cationic polymer of one embodiment may be present in an amount of from greater than 0% to about 5%, such as from about 0.25 to about 3% by weight, or from about 0.5 to about 1.5% by weight, based on the polymeric hair colorant composition in which it is present.

### EXAMPLES

### Example 1

Preparation of a Polyester Diisocyanate Moiety

In this example a castor-derived hydroxyl-terminated ricinoleate derivative is used as the diol. One equivalent of polycin D-265 (212 g) is combined with 2 equivalent of toluene diisocyanate (174 g) at room temperature (22° C.). The mixture is stirred at 100 revolutions per minute and the temperature monitored. The mixture will begin to heat up by exothermic reaction and no heat is to be applied to the reactor until the temperature in the reactor ceases to rise. Then the mixture temperature should be increased in 5° C. increments per ½ hour until the mixture reaches 60° C. The reaction should be continued until the % NCO=10.9% . The target % NCO is reached when every hydroxyl group in the mixture is reacted with an NCO group. Ideally, the result is a single diol endcapped with two diisocyanates. This outcome can be enhanced by slow addition of the diol to the diisocyanate. The addition should be in 10 g increments, added when the exotherm from the previous addition has ceased. However, chain extended variations of the above ideal outcome are useful, their primary disadvantage being that the product is slightly higher in viscosity. The ideal % NCO is calculated by dividing the weight of the functional isocyanate groups (2×42 Dalton) per product molecule by the total weight of the product molecule (424 Dalton+2×174 Dalton) yielding approximately 10.9%.

Alternatively, a lower molecular weight diol may be used, such as polycin D-290 where 1 equivalent of polycin D-290 is 193 g and the target % NCO is 84/(386+348)=11.4%.

Alternatively, a higher molecular weight diol may be used, such as polycin D-140 where 1 equivalent of polycin D-140 is 400 g and the target % NCO is 84/(800+348)=7.3%.

All polycin diols are available from Performance Materials (Greensboro, N.C.) and toluene diisocyanate is available from Sigma-Aldrich (Milwaukee, Wis.).

### Example 2

Preparation of a Polyether Diisocyanate Moiety

In this example a polyether hydroxyl-terminated copolymer of 75% ethylene oxide and 25% propylene oxide is used as the diol. One equivalent of UCON 75-H-450 (490 g) is combined with 2 equivalent of toluene diisocyanate (174 g) at room temperature (22° C.). The mixture is stirred at 100 revolutions per minute and the temperature monitored. The mixture will begin to heat up by exothermic reaction and no heat is to be applied to the reactor until the temperature in the reactor ceases to rise. Then the mixture temperature should be increased in 5° C. increments per ½ hour until the mixture reaches 60° C. The reaction should be continued until the % NCO=10.9% . The target % NCO is reached when every hydroxyl group in the mixture is reacted with an NCO group. Ideally, the result is a single diol endcapped with two diisocyanates. This outcome can be enhanced by slow addition of the diol to the diisocyanate. The addition should be in 10 g increments, added when the exotherm from the previous addition has ceased. However, chain extended variations of the above ideal outcome are useful, their primary disadvantage being that the product is slightly higher in viscosity. The ideal % NCO is calculated by dividing the weight of the functional isocyanate groups (2×42 Dalton) per product molecule by the total weight of the product molecule (980 Dalton+2×174 Dalton) yielding approximately 6.3%.

Polyether copolymers of ethylene oxide and propylene oxide diols are available from Dow Chemical (Midland, Mich.).

### Example 3

Preparation of a Polyester Triisocyanate

In this example a castor-derived hydroxyl-terminated ricinoleate derivative is used as the triol. One equivalent of polycin T-400 (141 g) is combined with 2 equivalent of toluene diisocyanate (174 g) at room temperature (22° C.). The mixture is stirred at 100 revolutions per minute and the temperature monitored. The mixture will begin to heat up by exothermic reaction and no heat is to be applied to the reactor until the temperature in the reactor ceases to rise. Then the mixture temperature should be increased in 5° C. increments per ½ hour until the mixture reaches 60° C. The reaction should be continued until the % NCO=13.3% . The target % NCO is reached when every hydroxyl group in the mixture is reacted with an NCO group. Ideally, the result is a single diol endcapped with two diisocyanates. This outcome can be enhanced by slow addition of the diol to the diisocyanate. The addition should be in 10 g increments, added when the exotherm from the previous addition has ceased. However, chain extended variations of the above ideal outcome are useful, their primary disadvantage being that the product is slightly higher in viscosity. The ideal % NCO is calculated by dividing the weight of the functional isocyanate groups (2×42 Dalton) per product molecule by the total weight of the product molecule (282 Dalton+2×174 Dalton) yielding approximately 13.3%.

The above reaction will yield a viscous product. A less viscous product can be obtained by adding propylene carbonate to the initial mixture. Additions up to 100% by weight of propylene carbonate are useful. Adjustment to the target NCO of the mixture must be performed using standard methods, or the propylene carbonate may be added after reaching the target % NCO.

Propylene carbonate is available from Sigma-Aldrich (Milwaukee, Wis.).

### Example 4

Preparation of a Polyether Triisocyanate

In this example a polyether hydroxyl-terminated copolymer of 75% ethylene oxide and 25% propylene oxide is used as the triol. One equivalent of Multranol 9199 (3066 g) is combined with 3 equivalent of toluene diisocyanate (261 g) at room temperature (22° C.). The mixture is stirred at 100 revolutions per minute and the temperature monitored. The mixture will begin to heat up by exothermic reaction and no heat is to be applied to the reactor until the temperature in the reactor ceases to rise. Then the mixture temperature should be increased in 5° C. increments per ½ hour until the mixture reaches 60° C. The reaction should be continued until the % NCO=1.3%. The target % NCO is reached when every hydroxyl group in the mixture is reacted with an NCO group. Ideally, the result is a single diol endcapped with two diisocyanates. This outcome can be enhanced by slow addition of the diol to the diisocyanate. The addition should be in 10 g increments, added when the exotherm from the previous addition has ceased. However, chain extended variations of the above ideal outcome are useful, their primary disadvantage being that the product is slightly higher in viscosity. The ideal % NCO is calculated by dividing the weight of the functional isocyanate groups (3×42 Dalton) per product molecule by the total weight of the product molecule (9199 Dalton+3×174 Dalton) yielding approximately 1.3%.

Multranol 9199 is available from Bayer (Pittsburg, Pa.).

### Example 5

Preparation of a Polyol Triisocyanate from Polyol Diol

Any of the diisocyanates prepared in Examples 1 and 2 trimerized by the addition of a low molecular weight triol such as polycin T-400 or trimethylolpropane (TMP). In this example TMP is used, but the method is adaptable to any triol. Complete trimerization of the diisocyanates of Example 1 and 2 will result in viscous products. To yield a lower viscosity product propylene carbonate can be employed or less triol can be used. In the later case, a mixture of diisocyanate and triisocyanate is obtained.

In this example the product of Example 2 is used as the polyether diisocyanate. One equivalent of Example 2 (682 g) is combined with 0.1 equivalent TMP (44.7 g) at room temperature (22° C.). The mixture is stirred at 100 revolutions per minute and the temperature monitored. The mixture will begin to heat up by exothermic reaction and no heat is to be applied to the reactor until the temperature in the reactor ceases to rise. Then the mixture temperature should be increased in 5° C. increments per ½ hour until the mixture reaches 60° C. The reaction should be continued until the % NCO=5.8%. The target % NCO is reached when every hydroxyl group in the mixture is reacted with an NCO group.

The ideal % NCO is calculated by dividing the weight fraction of the functional isocyanate groups 10%(3×42 Dalton) and 90%(2×42) per product molecule by the total weight fraction of the product molecule (3×1364 Dalton+134 Dalton)+1364 yielding approximately 0.3%+5.5%=5.8%.

TMP is available from Sigma-Aldrich (Milwaukee, Wis.).

### Example 6

Preparation of a Polymeric Hair Colorant using the Triisocyanate of Example 4

In this example the product of Example 4 is used as the polyether triisocyanate mixture. One hundred grams of Example 4 is combined with 1 g of chromophore at room temperature (22° C.) under nitrogen. The mixture is stirred at 100 revolutions per minute and the temperature monitored. The mixture will begin to heat up by exothermic reaction. When the temperature ceases to rise, a % NCO reading is taken. If % NCO>0 than an additional 1 g of chromophore extract is to be added. By a series of chromophore additions, one calculates the change in % NCO as a function of 1 g additions of chromophore extract, a linear plot is obtained from which the total amount of chromophore addition necessary to bring the % NCO to a target value is obtained. This amount of chromophore is added to the mixture and the mixture is reacted so that % NCO=target is obtained. For a tissue bonding aspect, % NCO must be greater than zero.

Alternatively, if the chromophore is a single known molecule, then theoretical amounts of chromophore may be used without the above described iterative steps. The above procedure pertains to chromophores derived from natural sources, such as botanicals.

### Example 7

Preparation of a polymeric hair colorant using the triisocyanate/diisocyanate of Example 5.

In this example the product of Example 5 is used as the polyether diisocyanate/triisocyanate mixture. One hundred grams of Example 5 is combined with 1 g of chromophore at room temperature (22° C.) under nitrogen. The mixture is stirred at 100 revolutions per minute and the temperature monitored. The mixture will begin to heat up by exothermic reaction. When the temperature ceases to rise, a % NCO reading is taken. If % NCO>0 than an additional 1 g of chromophore is to be added. By a series of chromophore addition one calculates the change in % NCO as a function of 1 g additions of chromophore, a linear plot is obtained from which the total amount of chromophore addition necessary to bring the % NCO to target is obtained. This amount of chromophore is added to the mixture and the mixture is reacted so that % NCO=target is obtained.\

### Example 8

Preparation of a highly-branched polymeric hair colorant with degradable links diol and triol can be combined to form a multi-branch polymer. In this instance, the Multranol 9199 triol is chain extended with polycin D-265 diol. The diisocyanate form of Example 2 is useful in chain extending the triisocyanate form of Example 4. The applicants wish to have on average 2 diisocyanates for every 3 triisocyanates, which forms a 5 armed isocyanate.

In this example 0.09 equivalents (292 g) of Example 4 is mixed with 0.04 equivalents (26.6 g) of Example 2. The triisocyanates of Example 4 and diisocyanates of Example 2 are chain extended with 0.08 equivalents lysine diamine to form a 5 armed isocyanate. One hundred grams of this reaction product is combined with 1 g of chromophore at room temperature (22° C.) under nitrogen. The mixture is stirred at 100 revolutions per minute and the temperature monitored. The mixture will begin to heat up by exothermic reaction. When the temperature ceases to rise, a % NCO reading is taken. If % NCO>0 than an additional 1 g of chromophore is to be added. By a series of chromophore addition, one calculates the change in % NCO as a function of lg additions of chromophore, a linear plot is obtained from which the total amount of chromophore addition necessary to bring the % NCO to target is obtained. This amount of chromophore is added to the mixture and the mixture is reacted so that % NCO=target is obtained.

Lysine diamine is available from Sigma-Aldrich (Milwaukee, Wis.).

### Example 9

Enhanced Hydrophilicity of Polymeric Hair Colorant

In this example the product of Example 4 is used as the polyether triisocyanate mixture. However, rather than using a poloxamer of propylene oxide and ethylene oxide, substitute a triol comprised entirely of ethylene oxide to obtain a modified Example 4. One hundred grams of modified Example 4 is combined with 1 g of chromophore at room temperature (22° C.) under nitrogen. The mixture is stirred at 100 revolutions per minute and the temperature monitored. The mixture will begin to heat up by exothermic reaction. When the temperature ceases to rise, a % NCO reading is taken. If % NCO>0 than an additional 1 g of chromophore is to be added. By a series of chromophore addition, one calculates the change in % NCO as a function of 1 g additions of chromophore, a linear plot is obtained from which the total amount of chromophore addition necessary to bring the % NCO to target is obtained. This amount of chromophore is added to the mixture and the mixture is reacted so that % NCO=target is obtained.

### Example 10

Polymeric hair colorant made more compatible with lipids by making the colorant more hydrophobic.

In this example, Example 9 is repeated using a modified Example 4 comprising a triol of only propylene oxide.

Thus, although there have been described particular embodiments of the present invention of a new and useful Polymeric Hair Color Composition it is not intended that such references be construed as limitations upon the scope of this invention except as set forth in the following claims.

What is claimed is:

1. A polymeric protein colorant composition comprising:
a polymeric backbone;
at least one urethane or urea link;
at least one chromophore; and
at least one pendant tissue bonding group.

2. The composition of claim 1 wherein the pendant tissue bonding group is an isocyanate or an amine.

3. The composition of claim 1 wherein the polymeric backbone is amphiphilic.

4. The composition of claim 3 wherein the polymeric backbone is a non-ionic triblock copolymer comprising one hydrophobic chain of polyoxypropylene (poly(propylene oxide)) and two hydrophilic chains of polyoxyethylene (poly(ethyleneoxide)).

5. The composition of claim 4 wherein the polymeric backbone comprises at least three hydroxyl (—OH) groups.

6. The composition of claim 1 wherein the polymeric backbone comprises at least one diol and at least one triol, the at least one diol and at least one triol linked to the polymeric backbone via urea or urethane links.

7. A polymeric protein colorant comprising the formula: 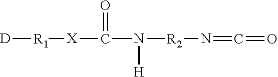
wherein (a) R.sub.1 selected from the group comprising C.sub.1-12 alkylen, C.sub.6-10 arylen, (C.sub.6-10) aryl-(C.sub.1-6) alkylen, and (C.sub.1-6) alkyl-(C.sub.6-10) arylen, -(C.sub.1-6 alkylen-O-).sub.n-C.-sub.1-6alkylen-; with “n” being an integer from 0 to 6; (b) X being oxygen or NR′ with R′ selected from the group comprising hydrogen, C.sub.1-6 alkyl, C.sub.6-10 aryl, (C.sub.6-10) aryl-(C.sub.1-6) alkyl, and (C.sub.1-6) alkyl-(C.sub.6-10) aryl; (c) R.sub.2 selected from the group comprising C.sub.1-12 alkylen, C.sub.5-6 cycloalkylen, C.sub.6-10 arylen, (C.sub.6-10) aryl-(C.sub.1-6) alkylen, and (C.sub.1-6) alkyl-(C.sub.6-10) arylen, -(C.sub.1-6alkylen-O-).sub.n-C.sub.1-6alkylen-; with “n” being an integer from 0 to 6; and (d) D is a dye moiety.

8. The composition of claim 7 wherein R.sub.1 and R.sub.2 have a chain length of C.sub.2-6.

9. The composition of claim 7 wherein D comprises the structure of FIG. 4, A comprising a substituted or unsubstituted fused aromatic or heterocyclic ring.

10. The composition of claim 9 wherein A is selected from the structures of FIG. 4a, FIG. 4b, and FIG. 4c, R.sub.3 is selected from the group comprising hydrogen, halogen, NR.sub.4R.sub.5, R.sub.50, and R.sub.5S; and for FIG. 4b, and FIG. 4c, Y is selected from the group comprising sulphur, oxygen, and NR.sub.4; wherein
R.sub.4 is selected from the group comprising hydrogen, C.sub.1-6 alkyl, C.sub.6-10 aryl, (C.sub.6-10) aryl-(C.sub.1-6) alkyl, and (C.sub.1-6) alkyl-(C.sub.6-10) aryl; and
R.sub.5 is selected from the group comprising C.sub.1-6 alkyl, C.sub.6-10 aryl, (C.sub.6-10) aryl-(C.sub.1-6) alkyl, and (C.sub.1-6) alkyl-(C.sub.6-10) aryl.

11. The composition of claim 10 wherein the R.sub.4 and R.sub.5 independently for each further comprise hydroxyl, C.sub.1-6 alkoxyl, C.sub.6-10 aryloxy, or halogen.

12. The composition of claim 7 wherein D comprises the structure of FIG. 5.

13. The composition of claim 7 wherein D comprises the structure of FIG. 6, Ring B being annelated in the 3,4-position with a group selected from: —NR.sub.6(CO).sub.m-NR.sub.7-, or —O—CO—NR.sub.6-; wherein “m” is 1 or 2; wherein
R.sub.6 and R.sub.7 are independently hydrogen, C.sub.1-6 alkyl, C.sub.6-10 aryl, (C.sub.6-10) aryl-(C.sub.1-6)alkyl, or (C.sub.1-6)alkyl-(C.sub.6-10)aryl.

14. The composition of claim 13 wherein R.sub.6 and R.sub.7 independently for each further comprise amino, C.sub.1-6 alkylamino, C.sub.6-10 cycloalkylamino, hydroxyl, C.sub.1-6 alkoxyl, C.sub.6-10 aryloxy or halogen.

15. The composition of claim 7 wherein D comprises the structure of FIG. 7, R.sub.3 is selected from the group comprising hydrogen, halogen, NR.sub.4R.sub.5, R.sub.50, and R.sub.5S; wherein R.sub.4 is selected from the group comprising hydrogen, C.sub.1-6 alkyl, C.sub.6-10 aryl, (C.sub.6-10) aryl-(C.sub.1-6) alkyl, and (C.sub.1-6) alkyl-(C.sub.6-10) aryl; and R.sub.5 is selected from the group comprising C.sub.1-6 alkyl, C.sub.6-10 aryl, (C.sub.6-10) aryl-(C.sub.1-6) alkyl, and (C.sub.1-6) alkyl-(C.sub.6-10) aryl.

16. The composition of claim 15 wherein the R.sub.4 and R.sub.5 independently for each further comprise hydroxyl, C.sub.1-6 alkoxyl, C.sub.6-10 aryloxy, or halogen.

17. The composition of claim 7 wherein D comprises the structure of FIG. 8, R.sub.3 is selected from the group comprising hydrogen, halogen, NR.sub.4R.sub.5, R.sub.50, and R.sub.5S; wherein R.sub.4 is selected from the group comprising hydrogen, C.sub.1-6 alkyl, C.sub.6-10 aryl, (C.sub.6-10) aryl-(C.sub.1-6) alkyl, and (C.sub.1-6) alkyl-(C.sub.6-10) aryl; and R.sub.5 is selected from the group comprising C.sub.1-6 alkyl, C.sub.6-10 aryl, (C.sub.6-10) aryl-(C.sub.1-6) alkyl, and (C.sub.1-6) alkyl-(C.sub.6-10) aryl.

18. The composition of claim 17 wherein the R.sub.4 and R.sub.5 independently for each further comprise hydroxyl, C.sub.1-6 alkoxyl, C.sub.6-10 aryloxy, or halogen.

19. A method for the preparation of the composition according to claim 7 comprising the steps of:
(a) reacting a hydroxy or amino functionalized dye moiety with a diisocyanate moiety wherein R.sub.2″ is selected from the group comprising C.sub.1-12 alkylen, C.sub.5-6 cycloalkylen, C.sub.6-10 arylen, (C.sub.6-10) aryl-(C.sub.1-6) alkylen, and (C.sub.1-6) alkyl-(C.sub.6-10) arylen, —(C.sub.1-6alkylen-O-).sub.n-C.sub.1-6alkyl-en-; with “n” being a number from 0 to 6.

20. The method of claim 19, wherein the diisocyanate moiety is selected from the group comprising C.sub.6-12 alkylendiisocyanate, toluylene-2,4-diisocyanate, and 1-isocyanato-3-isocyanatoomethyl-3,5,5-trimethyl-cyclohexane.
